(19) United States
(12) Patent Application Publication
(10) Pub. No.: US 2023/0277686 A1
(43) Pub. Date: Sep. 7, 2023

(54) A NOVEL MUSCLE-SPECIFIC PROMOTER

(71) Applicants: GENETHON, Evry-Courcouronnes (FR); INSERM (INSTITUT NATIONAL DE LA SANTE ET DE LA RECHERCH MEDICALE), Paris (FR); UNIVERSITE D'EVRY-VAL-D'ESSONNE, Evry-Courcouronnes (FR)

(72) Inventors: William LOSTAL, Savigny Sur Orge (FR); Isabelle RICHARD, Corbeil Essonnes (FR)

(21) Appl. No.: 18/004,372

(22) PCT Filed: Jul. 9, 2021
(86) PCT No.: PCT/EP2021/069133

§ 371 Date: Jan. 5, 2023

(30) Foreign Application Priority Data
Jul. 10, 2020 (EP) 20305796.3

(51) Int. Cl. A61K 48/00; C12N 15/86; C12N 9/64; C07K 14/47; A61P 21/00

(52) U.S. Cl. CPC A61K 48/0058; C12N 15/86; C12N 9/6472; C12Y 304/22054; C07K 14/4708; C07K 14/47; C07K 14/471; A61P 21/00; C12N 2750/14143; C12N 2820/007; C07K 2319/00

(57) ABSTRACT

The present invention concerns a novel short promoter characterized by a high activity in the skeletal muscles and a low activity in the heart. It then constitutes a valuable candidate especially for driving the expression of transgenes encoding proteins useful for the treatment of muscular dystrophies.

The present invention is based on the identification of a novel promoter having a small size and an expression profile of interest, i.e. a high activity in the skeletal muscles and a low activity in the heart. It then offers a valuable and safe therapeutic tool, for example for driving the expression of transgenes encoding proteins useful for the treatment of muscular dystrophies.

### BACKGROUND OF THE INVENTION

Muscular dystrophy (MD) is a group of muscle diseases that results in increasing weakening and breakdown of skeletal muscles over time. The disorders differ in which muscles are primarily affected, the degree of weakness, how fast they worsen, and when symptoms begin. Many people will eventually become unable to walk. Some types are also associated with problems in other organs. In some cases of muscular dystrophy, cardiomyopathy is also observed.

The muscular dystrophy group contains thirty different genetic disorders that are usually classified into nine main categories or types. The most common type is Duchenne muscular dystrophy (DMD), which typically affects males beginning around the age of four. Other types include Becker muscular dystrophy, facioscapulohumeral muscular dystrophy, limb-girdle muscular dystrophy (LGMD), and myotonic dystrophy. They are due to mutations in genes that are involved in making muscle proteins. This can occur due to either inheriting the defect from one's parents or the mutation occurring during early development. Disorders may be X-linked recessive, autosomal recessive, or autosomal dominant. Diagnosis often involves blood tests and genetic testing.

There is no cure for muscular dystrophy. Physical therapy, braces, and corrective surgery may help with some symptoms. Assisted ventilation may be required in those with weakness of breathing muscles. Medications used include steroids to slow muscle degeneration, anticonvulsants to control seizures and some muscle activity, and immunosuppressants to delay damage to dying muscle cells. Outcomes depend on the specific type of disorder.

Gene therapy, as a treatment, is in the early stages of study in humans. It is generally based on the systemic administration of an expression system harboring a transgene encoding the wild type protein which is mutated in the subject to be treated.

In relation to gene therapy, a safe expression system is defined as one which ensures the production of a therapeutically effective amount of the protein in the target tissues, i.e. in the tissues wherein said protein is needed to cure the abnormalities linked to the deficiency of the native protein, without displaying any toxicity, especially in the essential and vital organs or tissues. In relation to muscular dystrophy, target tissues concern the skeletal muscles and possibly the heart.

The tight regulation of the expression of the transgene appears as a key element. As an example, document WO2014/167253 has reported that the systemic administration of an AAV vector comprising a nucleic acid sequence encoding myotubularin (to treat X-linked myotubular myopathy or XLMTM) or calpain 3 (to treat Limb-Girdle Muscular Dystrophy type 2A or LGMD2A) placed under the control of the human desmin promoter leads to a muscular rescue but is associated with cardiac toxicity.

A number of promoters efficient for high muscle expression exist. As an example, Brennan et al. (The Journal of Biological Chemistry, 1993, 268(1): 719-25) have reported that the −2000 to +239 region of the human alpha actin ACTA1 gene (SEQ ID NO: 1) allows a high-level expression in adult skeletal muscles, as well as a striated muscle-specific expression and a correct regulation during development. However, the large size of such a promoter (2239 nucleotides) renders it incompatible with a large number of applications.

In relation to the skeletal muscle alpha actin gene promoter region:

Muscat et al. (Mol. Cell. Biol., 1987, 7(11): 4089-99) have identified multiple 5′-flanking regions of the human gene which synergically modulate muscle-specific expression.

Petropoulos et al. (Mol. Cell. Biol., 1989, 9(9): 3785-92) have studied the expression profile of the promoter of the chicken gene.

Muscat et al. (Gene Expression, 1992, 2(2): 111-126) have identified a muscle-specific enhancer in the human skeletal alpha actin gene.

Besides and as shown in the present application in relation to the desmin promoter, most of them also display a high activity in the heart, possibly associated with cardiac toxicity. Moreover, it is commonly observed that the smaller the promoter, the more difficult to obtain a specificity of expression. As an example, a small promoter with high muscle expression is the synthetic C5-12 promoter (361 nucleotides) which was shown to be leaky in a number of non-muscle cells.

Therefore, there is still a need in the art to design transcriptional elements as small as possible leading to high level and specific expression of genes in skeletal muscle cells, in order to achieve therapeutic levels of protein expression and to avoid the potential side effects inherent to a widespread gene expression.

### BRIEF SUMMARY OF THE INVENTION

The present invention aims at providing synthetic promoters, which are muscle-specific, i.e. having an expression activity higher in the skeletal muscles than in all the other tissues or organs, especially the heart, while being of small size in order to be compatible with any expression system, especially adeno-associated (AAV) vectors.

These novel promoters may be used in gene therapy, e.g. for the treatment of neuromuscular diseases including muscular dystrophies.

The invention relates to a nucleic acid molecule comprising such a synthetic promoter.

The promoter of the invention may be operably linked to a transgene of interest. Accordingly, the invention further relates to an expression cassette comprising the nucleic acid molecule described herein, operably linked to a transgene.

The invention further relates to a vector comprising the expression cassette described above. In a particular embodiment, the vector is a plasmid vector. In another embodiment, the vector is a viral vector.

The invention also relates to an isolated recombinant cell comprising the nucleic acid construct according to the invention.

The invention further relates to a pharmaceutical composition comprising, in a pharmaceutically acceptable carrier, the vector or the isolated cell of the invention.

Furthermore, the invention also relates to the expression cassette, the vector or the cell disclosed herein, for use as a medicament. In this aspect, the transgene of interest comprised in the expression cassette, the vector or the cell is a therapeutic transgene.

The invention further relates to the expression cassette, the vector or the cell disclosed herein, for use in gene therapy.

In another aspect, the invention relates to the expression cassette, the vector or the cell disclosed herein, for use in the treatment of a neuromuscular disorder, e.g. a muscular dystrophy.

### Definitions

Unless otherwise defined, all technical and scientific terms used therein have the same meaning as commonly understood by one of ordinary skill in the art. The terminology used in the description is for the purpose of describing particular embodiments only and is not intended to be limiting.

The articles “a” and “an” are used herein to refer to one or to more than one (i.e., to at least one) of the grammatical object of the article. By way of example, “an element” means one element or more than one element.

“About” or “approximately” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

A “nucleotide sequence encoding an amino acid sequence” includes all nucleotide sequences that are degenerate versions of each other and that encode the same amino acid sequence. The phrase nucleotide sequence that encodes a protein or a RNA or a cDNA may also include introns to the extent that the nucleotide sequence encoding the protein may in some version contain an intron(s).

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

A protein may be “altered” and contain deletions, insertions, or substitutions of amino acid residues which produce a silent change and result in a functionally equivalent. Deliberate amino acid substitutions may be made on the basis of similarity in polarity, charge, solubility, hydrophobicity, hydrophilicity, and/or the amphipathic nature of the residues as long as the biological activity is retained. For example, negatively charged amino acids may include aspartic acid and glutamic acid; positively amino acids may include lysine and arginine; and amino acids with uncharged polar head groups having similar hydrophilicity values may include leucine, isoleucine, and valine, glycine and alanine, asparagine and glutamine, serine and threonine, and phenylalanine and tyrosine.

A “variant”, as used herein, refers to an amino acid sequence that is altered by one or more amino acids. The variant may have “conservative” changes, wherein a substituted amino acid has similar structural or chemical properties, e. g. replacement of leucine with isoleucine. A variant may also have “non-conservative” changes, e. g. replacement of a glycine with a tryptophan. Analogous minor variations may also include amino acid deletions or insertions, or both. Guidance in determining which amino acid residues may be substituted, inserted, or deleted without abolishing biological or immunological activity may be found using computer programs well known in the art.

“Identical” or “homologous” refers to the sequence identity or sequence similarity between two polypeptides or between two nucleic acid molecules. When a position in both of the two compared sequences is occupied by the same base or amino acid monomer subunit, e.g., if a position in each of two DNA molecules is occupied by adenine, then the molecules are homologous or identical at that position. The percent of homology/identity between two sequences is a function of the number of matching positions shared by the two sequences divided by the number of positions compared ×100. For example, if 6 of 10 of the positions in two sequences are matched then the two sequences are 60% identical. Generally, a comparison is made when two sequences are aligned to give maximum homology/identity.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like. “Viral vector” refers to a non-replicating, non-pathogenic virus engineered for the delivery of genetic material into cells. In viral vectors, viral genes essential for replication and virulence are replaced with an expression cassette for the gene of interest. Thus, the viral vector genome comprises the expression cassette flanked by the viral sequences required for viral vector production.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

The term “promoter” as used herein is defined as a nucleic acid sequence recognized by the transcriptional machinery of the cell, or introduced transcriptional machinery, required to initiate the specific transcription of a polynucleotide sequence, in particular of a gene of interest.

As used herein, the term “promoter/regulatory sequence” means a nucleic acid sequence, which is required for expression of a gene product (i.e. the gene of interest) operably linked to the promoter/regulatory sequence. In some instances, this sequence may be the core promoter sequence and in other instances, this sequence may also include an enhancer sequence and other regulatory elements, which are required for expression of the gene product. The promoter/regulatory sequence may, for example, be one, which expresses the gene product in a tissue specific manner.

The term “operably linked” as used above refers to the juxtaposition of the gene of interest with the sequences controlling its transcription. There may be additional residues between the promoter and the gene of interest so long as this functional relationship is preserved.

A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifies a gene product, causes the gene product to be produced in a cell substantially only when an inducer which corresponds to the promoter is present in the cell or when a repressor thereof is removed.

A “tissue-specific” promoter is a nucleotide sequence which, when operably linked with a polynucleotide encodes or specified by a gene, causes the gene product to be produced in a cell preferentially if the cell is a cell of the tissue type corresponding to the promoter.

A “gene of interest” or “transgene” is a gene useful for a particular application, such as with no limitation, diagnosis, reporting, modifying, therapy and genome editing. For example, the gene of interest may be a therapeutic gene, a reporter gene or a genome-editing enzyme. In some embodiments, the gene of interest is a human gene.

In some embodiments, the gene of interest is a functional version of a gene or a fragment thereof. The functional version of said gene includes the wild-type gene, a variant gene such as variants belonging to the same family and others, or a truncated version, which preserves the functionality of the encoded protein at least partially. A functional version of a gene is useful for replacement or additive gene therapy to replace a gene, which is deficient or non-functional in a patient. In other embodiments, the gene of interest is a gene which inactivates a dominant allele causing an autosomal dominant genetic disease. A fragment of a gene is useful as recombination template for use in combination with a genome editing enzyme.

Alternatively, the gene of interest may encode a protein of interest for a particular application (for example an antibody or antibody fragment, a genome-editing enzyme) or a RNA. In some embodiments, the protein is a therapeutic protein including a therapeutic antibody or antibody fragment, or a genome-editing enzyme. In some embodiments, the RNA is a therapeutic RNA. The gene of interest is a functional gene able to produce the encoded protein, peptide or RNA in the target cells of the disease, in particular muscle cells. The RNA is advantageously complementary to a target DNA or RNA sequence or binds to a target protein. For example, the RNA is an interfering RNA such as a shRNA, a microRNA, a guide RNA (gRNA) for use in combination with a Cas enzyme or similar enzyme for genome editing, an antisense RNA capable of exon skipping such as a modified small nuclear RNA (snRNA) or a long non-coding RNA. The interfering RNA or microRNA may be used to regulate the expression of a target gene involved in muscle disease. The guide RNA in complex with a Cas enzyme or similar enzyme for genome editing may be used to modify the sequence of a target gene, in particular to correct the sequence of a mutated/deficient gene or to modify the expression of a target gene involved in a disease, in particular a neuromuscular disease. The antisense RNA capable of exon skipping is used in particular to correct a reading frame and restore expression of a deficient gene having a disrupted reading frame. In some embodiments, the RNA is a therapeutic RNA.

In some embodiments of the invention, said gene of interest encodes a therapeutic protein or therapeutic ribonucleic acid that can be any protein or ribonucleic acid providing a therapeutic effect to skeletal muscular cells. In another embodiment of the invention, the said therapeutic protein can be any protein providing a therapeutic effect outside of the skeletal muscular cells. The said therapeutic protein can indeed be secreted by the said cells.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics, which are normal or expected for one cell or tissue type, might be abnormal for a different cell or tissue type.

The terms “patient,” “subject,” “individual,” and the like are used interchangeably herein, and refer to any animal, or cells thereof whether in vitro or in situ, amenable to the methods described herein. A subject can be a mammal, e.g. a human, a dog, but also a mouse, a rat or a nonhuman primate. In certain non-limiting embodiments, the patient, subject or individual is a human.

A “disease” or a “pathology” is a state of health of a subject wherein the subject cannot maintain homeostasis, and wherein if the disease is not ameliorated then the subject's health continues to deteriorate. In contrast, a “disorder” in a subject is a state of health in which the subject is able to maintain homeostasis, but in which the subject's state of health is less favorable than it would be in the absence of the disorder. Left untreated, a disorder does not necessarily cause a further decrease in the subject's state of health.

A disease or disorder is “alleviated” or “ameliorated” if the severity of a symptom of the disease or disorder, the frequency with which such a symptom is experienced by a patient, or both, is reduced. This also includes halting progression of the disease or disorder. A disease or disorder is “cured” if the severity of a symptom of the disease or disorder, the frequency with which such a symptom is experienced by a patient, or both, is eliminated.

A “therapeutic” treatment is a treatment administered to a subject who exhibits signs of pathology, for the purpose of diminishing or eliminating those signs. A “prophylactic” treatment is a treatment administered to a subject who does not exhibit signs of pathology or has not be diagnosed for the pathology yet, for the purpose of preventing or postponing the occurrence of those signs.

As used herein, “treating a disease or disorder” means reducing the frequency or severity of at least one sign or symptom of a disease or disorder experienced by a subject. Disease and disorder are used interchangeably herein in the context of treatment.

An “effective amount” of a compound is that amount of compound which is sufficient to provide a beneficial effect to the subject to which the compound is administered. The phrase “therapeutically effective amount”, as used herein, refers to an amount that is sufficient or effective to prevent or treat (delay or prevent the onset of, prevent the progression of, inhibit, decrease or reverse) a disease or condition, including alleviating symptoms of such diseases. An “effective amount” of a delivery vehicle is that amount sufficient to effectively bind or deliver a compound.

### DETAILED DESCRIPTION OF THE INVENTION

This invention is based on the identification of promoters derived from the human ACTA1 gene.

The product encoded by the ACTA1 gene (also named actin alpha 1, skeletal muscle or HAS for the human version) belongs to the actin family of proteins, which are highly conserved proteins that play a role in cell motility, structure and integrity. Alpha, beta and gamma actin isoforms have been identified, with alpha actins being a major constituent of the contractile apparatus, while beta and gamma actins are involved in the regulation of cell motility. This actin is an alpha (a) actin that is found in skeletal muscle. The gene is located at position 1q42.13 (NCBI Reference Sequence: NG 006672.1).

As demonstrated in the present application, it is possible to have a sequence of 324 nucleotides or even smaller, which has an activity in the skeletal muscles higher than in any other tissue or organ, especially the heart. As a result, such a promoter can have a muscle activity higher than the well-known human desmin promoter of 1061 nucleotides, with a lower activity in the heart.

According to one aspect, the present invention concerns a promoter comprising a proximal region and a distal region derived from the human ACTA1 gene, as defined below.

The nucleotide positions referenced in the present application for the cited promoters are numbered relative to the presumed transcription initiation site (or cap site representing position +1) of the (native) gene concerned, advantageously of the human ACTA1 gene. By way of illustration, the first nucleotide directly upstream from the transcription initiation site is numbered −1 whereas the nucleotide following it is numbered +2.

In the frame of the application, the proximal region is the region located just upstream the 5′ end of the gene of interest, e.g. the start codon of an encoding sequence. The distal region is located remotely, i.e. upstream the proximal region. In other words, the proximal region is nearer to the gene to be expressed than is the distal region.

According to a specific embodiment, the proximal region of the promoter according to the invention comprises the core or basal promoter of the ACTA1 gene, sufficient to ensure a minimal level of transcription.

According to one embodiment, the promoter of the invention comprises the proximal region operably linked with the distal region.

In that context, “operably linked” refers to a juxtaposition of the proximal and distal regions permitting them to mediate expression of a gene of interest placed under the control of said promoter. For instance, the distal region is operably linked to the proximal region if it enhances transcription of the gene of interest, resulting in an enhancement of its expression in the host cell or organism. There may be additional residues between the proximal region and the distal region so long as this functional relationship is preserved.

Advantageously, the distal region is operably linked with the proximal region if the distal region increases gene expression driven by the proximal region. The distal region may be adjacent, at a close distance or over a distance of up to several kb to the proximal region. Advantageously, the distal region is positioned upstream of the proximal region, more advantageously with a distance separating said two regions by less than 500 bp, preferably less than 200 bp. More preferably, the distal region is immediately adjacent or attached to the proximal region.

According to various embodiments, said distal and proximal regions can be contiguous or can be separated by a sequence, possibly from the human ACTA1 promoter, advantageously a sequence naturally surrounding said regions, but can also be an exogenous sequence or a spacer. According to a specific embodiment, the sequence separating the distal and proximal regions has a size of less than 500, 450, 400, 350, 300, 250, 200, 150, or even less than 100 or 50 nucleotides.

Moreover, the orientation of the distal region may be sense (5′→3′) or antisense (3′→5) relative to the transcriptional direction conferred by the proximal region. The optimal location and orientation of each element present in the promoter of the present invention relative to the others can be determined by routine experimentation.

According to one embodiment, the distal region comprises or consists of the −1282 to −1177 region of the human ACTA1 gene. In other words, it comprises or consists of a nucleotide sequence as shown in SEQ ID NO: 1 from positions 719 to 824 or a nucleotide sequence as shown in SEQ ID NO: 3.

According to other embodiments, the distal region can comprise or consist of:
The sequence SEQ ID NO: 3 extended in 5′ and/or in 3′ up to 10 nucleotides, i.e. possibly corresponding to positions 709 to 834 of SEQ ID NO: 1;
The sequence SEQ ID NO: 3 truncated in 5′ and/or in 3′ up to 10 nucleotides, i.e. possibly corresponding to positions 729 to 814 of SEQ ID NO: 1 (also corresponding to positions 11 to 96 of SEQ ID NO: 3);
A sequence having identity greater than or equal to 90%, preferably greater than or equal to 95% or even 99% with said sequences, advantageously with SEQ ID NO: 3, especially in order to cover the corresponding sequences but from another origin, for example from chicken, mouse or rat, or even derivatives thereof as long as the said sequences have the same activity, advantageously the same expression profile as e.g. SEQ ID NO: 3.

According to a specific embodiment, the distal region comprises or consists of the reverse sequence of the sequences as disclosed above. In the frame of the present application, the reverse sequence means the same sequence but in the opposite direction or in the antisense orientation.

According to one embodiment, the proximal region comprises or consists of the −153 to +1 region of the human ACTA1 gene. In other words, it comprises or consists of a nucleotide sequence as shown in SEQ ID NO: 1 from positions 1848 to 2001 or a nucleotide sequence as shown in SEQ ID NO: 4 from positions 39 to 192.

According to other embodiments, the proximal region can comprise or consist of:
The sequence SEQ ID NO: 1 from positions 1848 to 2001, extended in 5′ up to position 1810 of SEQ ID NO: 1 or even up to position 1729 of SEQ ID NO: 1 and/or extended in 3′ up to position 2027 of SEQ ID NO: 1 or even to position 2239 of SEQ ID NO: 1;
A sequence having identity greater than or equal to 90%, preferably greater than or equal to 95% or even 99% with said sequences, advantageously with SEQ ID NO: 4, especially in order to cover the corresponding sequences but from another origin, for example from chicken, mouse or rat, or even derivatives thereof as long as the said sequences have the same activity, advantageously the same expression profile as e.g. SEQ ID NO: 4.

According to a specific embodiment, the proximal region has the sequence SEQ ID NO: 4.

According to another specific embodiment, the sequence corresponding to SEQ ID NO: 1 from positions 1848 to 1914 is present in the proximal region in the antisense orientation.

According to a further embodiment, the proximal region of a promoter according to the invention comprises or consists of the −98 to +1 region of the human ACTA1 gene. In other words, it comprises or consists of a nucleotide sequence as shown in SEQ ID NO: 1 from positions 1903 to 2001 or a nucleotide sequence as shown in SEQ ID NO: 4 from positions 94 to 192.

According to one embodiment of the invention, the promoter has a length of less than 800, 750, 700 nucleotides, advantageously of less than 650, 600, 550, 500, 450, 400 or 350 nucleotides. In another particular embodiment, the promoter has a size of at least 150, 200, 250 or 300 nucleotides.

Advantageously, the promoter sequence has a size between 250 and 350 nucleotides, advantageously between 260 and 325 nucleotides.

According to a specific embodiment, the promoter according to the invention comprises the sequences SEQ ID NO: 3 and SEQ ID NO: 4.

According to one embodiment, the promoter according to the invention comprises SEQ ID NO: 3 attached to SEQ ID NO: 4, i.e. SEQ ID NO: 2.

According to another embodiment, the promoter according to the invention comprises the reverse sequence of SEQ ID NO: 3 attached to SEQ ID NO: 4.

According to a specific embodiment, the promoter according to the invention comprises or consists of the sequence SEQ ID NO: 2 or a sequence having identity greater than or equal to 90%, preferably greater than or equal to 95% or even 99% with SEQ ID NO: 2, especially in order to cover derivatives thereof as long as the said sequences have the same activity, advantageously the same expression profile as e.g. SEQ ID NO: 2. According to a more specific embodiment, the promoter according to the invention comprises or consists of a sequence having identity greater than or equal to 90%, preferably greater than or equal to 91%, or 92% or 93% or 94% or 95% or 96% or 97% or 98% or 99% with SEQ ID NO: 2.

A sequence having identity greater than or equal to 90% with SEQ ID NO: 2 covers inter alia:
the corresponding sequences but from another origin, for example from chicken, mouse or rat;
SEQ ID NO: 2 extended or truncated in 5′ up to 10 nucleotides and/or extended or truncated in 3′, e.g. ending at position 298 of SEQ ID NO:2.

According to the invention, the promoter is a myogenic promoter, advantageously a muscle-specific promoter, i.e. has an activity in the muscles, advantageously in the skeletal muscles, higher than in any other tissue or organ, especially the heart. Advantageously, the promoter according to the invention ensures or allows an expression level in the skeletal muscles higher than in the heart.

In other words and notably in relation to muscular dystrophy, the promoter of the invention can allow:

the expression at a therapeutically acceptable level of a protein of interest in the target tissue(s), advantageously in the skeletal muscles and possibly in the heart; but the expression at an adequate level of said protein in the heart compared to its expression level in the target tissues, especially in the skeletal muscles, so as to avoid any potential cardiac toxicity (i.e. at a therapeutically acceptable level).

In the frame of the application, a target tissue is defined as a tissue or organ in which the protein is to play a therapeutic role, especially in cases where the native gene encoding this protein is defective. According to a particular embodiment of the invention, the target tissue includes the striated skeletal muscles, hereafter referred to as skeletal muscles, i.e. all the muscles involved in motor ability and the diaphragm, and possibly smooth muscles. Non limiting examples of target skeletal muscles are tibialis anterior (TA), gastrocnemius, soleus, quadriceps, psoas, deltoid, diaphragm, gluteus, extensorum digitorum longus (EDL), biceps brachii muscles, . . . . As explained above and in relation with some muscular dystrophies, the heart can also be a target tissue but wherein an excessive level or activity of protein can be toxic.

In the context of the invention, the term “therapeutically acceptable level” refers to the fact that the protein produced helps improve the pathological condition of the patient, particularly in terms of quality of life or lifespan. Thus and in connection with a disease affecting skeletal muscles, this involves improving the muscular condition of the subject affected by the disease or restoring a muscular phenotype similar to that of a healthy subject. The muscular state, mainly defined by the strength, size, histology and function of the muscles, can be evaluated by different methods known in the art, e.g. biopsy, measurement of the strength, muscle tone, volume, or mobility of muscles, clinical examination, medical imaging, biomarkers, etc.

Thus, the criteria that help assess a therapeutic benefit as regards skeletal muscles and that can be evaluated at different times after the treatment are in particular at least one among:
increased life expectancy;
increased muscle strength;
improved histology; and/or
improved functionality of the diaphragm.

In the context of the invention, the term “toxically acceptable level” refers to the fact that the protein produced from the expression system does not cause significant alteration of the tissue, especially histologically, physiologically and/or functionally. In particular, the expression of the protein may not be lethal. The toxicity in a tissue can be evaluated histologically, physiologically and functionally.

In relation to heart, any toxicity of a protein can be evaluated by a study of the morphology and the heart function, by clinical examination, electrophysiology, imaging, biomarkers, monitoring of the life expectancy or by histological analysis, including the detection of fibrosis and/or cellular infiltrates and/or inflammation, for example by staining with sirius red or hematoxyline (e.g. Hematoxyline-Eosin-Saffran (HES) or Hematoxyline-Phloxin-Saffron (HFS)).

According to a specific embodiment, the expression profile of a promoter according to the invention can be evaluated by calculating the ratio between the amount in the skeletal muscles, e.g. in the TA muscle, of the expressed gene and the amount in the heart of the expressed gene. Advantageously, this ratio (relative activity in the skeletal muscles vs in the heart) is superior or equal to 1, 5, 10, or even 20, 30, 40, 50, 60, 70, 80, 90 or even 100.

The evaluation of the amount of protein produced from the gene operably linked to the promoter of the invention can be carried out by immunodetection using an antibody directed against said protein, for example by Western blot or ELISA, or by mass spectrometry. Alternatively, the corresponding messenger RNAs may be quantified, for example by PCR or RT-PCR. This quantification can be performed on one sample of the tissue or on several samples. Thus and in the case where the target tissues are skeletal muscles, it may be carried out on a muscular type or several types of muscles (for example quadriceps, diaphragm, tibialis anterior, triceps, etc.).

Advantageously, the promoter of the invention is more active than the reference desmin promoter (advantageously of sequence SEQ ID NO: 5) in the skeletal muscles. In other words, the muscular level of expression of any transgene operably linked and placed under the control of the promoter of the invention is advantageously higher than the one obtained when said transgene is operably linked to the desmin promoter.

Advantageously, the promoter of the invention is less active than the reference desmin promoter (advantageously of sequence SEQ ID NO: 5) in the heart. In other words, the cardiac level of expression of any transgene operably linked and placed under the control of the promoter of the invention is advantageously lower than the one obtained when said transgene is operably linked to the desmin promoter.

As a consequence, a promoter according to the invention displays a very high activity in the skeletal muscles. Moreover, it avoids the potential cardiac toxicity linked to the other available promoters having a high activity in the skeletal muscles.

According to another embodiment, the promoter according to the invention allows a low expression in non-target tissues, i.e. in the tissues in which the protein encoded by the transgene operably linked to the promoter has no therapeutic effect or in which said protein is not naturally expressed. As mentioned above, skeletal muscles and possibly heart are advantageously not considered as non-target tissues. On the contrary, the liver, the kidneys, the brain and the adrenal glands can be considered as non-target tissues.

According to a further specific embodiment, the promoter according to the invention has an activity in non-target tissues, especially the liver, kidney and adrenal glands, lower than in the skeletal muscles and possibly lower than in the heart.

According to another aspect, the present invention concerns a nucleic acid comprising a promoter as disclosed above.

According to another aspect, the present invention concerns an expression system comprising a gene of interest or a transgene placed under the control of a promoter as disclosed above.

In the frame of the invention, an expression system is generally defined as a polynucleotide which allows the in vivo production of a gene of interest, possibly a protein. According to one aspect, said system comprises a nucleic acid encoding said protein, also named a transgene, and at least a promoter according to the invention. Said expression system can then corresponds to an expression cassette. Alternatively, said expression cassette can be harboured by a vector or a plasmid. The wording “expression system” as used therein covers all aspects.

Suitably, an expression system of the invention comprises a promoter as defined above governing the transcription of the sequence encoding the protein, preferably placed at 5′ of said sequence and functionally linked thereto. Preferably, this ensures a therapeutically acceptable level of expression of the protein in the skeletal muscles and in the heart, as well as a toxically acceptable level in the heart, as defined above.

According to a one embodiment, the expression system of the invention comprises a sequence encoding a protein of interest, advantageously a protein having a therapeutic activity in the skeletal muscles and possibly in the heart, corresponding to a transgene. In the context of the invention, the term “transgene” refers to a sequence, preferably an open reading frame, provided in trans using the expression system of the invention. The concept of therapeutic activity is defined as above in connection with the term “therapeutically acceptable level”.

According to a particular embodiment, this sequence is a copy, identical or equivalent, of an endogenous sequence present in the genome of the body into which the expression system is introduced.

According to another embodiment, the endogenous sequence has one or more mutations rendering the protein partially or fully non-functional or even absent (lack of expression or activity of the endogenous protein), or not properly located in the desired subcellular compartment. In other words, the expression system of the invention is intended to be administered to a subject having a defective copy of the sequence encoding the protein and having an associated pathology. In this context, the protein encoded by the sequence carried by the expression system of the invention can therefore be defined as a protein whose mutation causes e.g. a muscular dystrophy.

As already stated, a promoter according to the invention can be used to produce any protein of interest, especially any therapeutic protein.

Of particular interest is the use of a promoter according to the invention in an expression system dedicated to the treatment of neuromuscular diseases, especially muscular dystrophies.

Examples of mutated genes involved in neuromuscular genetic disorders of interest, and then examples of genes that can be placed under the control of a promoter according to the invention, are listed below:

Muscular Dystrophies

| Gene | Protein |
|---|---|
| DMD | Dystrophin |
| EMD | Emerin |
| FHL1 | Four and a half LIM domain 1 |
| LMNA | Lamin A/C |
| SYNE1 | Spectrin repeat containing, nuclear envelope 1 (nesprin 1) |
| SYNE2 | Spectrin repeat containing, nuclear envelope 2 (nesprin 2) |
| TMEM43 | Transmembrane protein 43 |
| TOR1AIP1 | Torsin A interacting protein 1 |
| DUX4 | Double homeobox 4 |
| SMCHD1 | Structural maintenance of chromosomes flexible hinge |
| | domain containing 1 |
| PTRF | Polymerase I and transcript release factor |
| MYOT | Myotilin |
| CAV3 | Caveolin 3 |
| DNAJB6 | HSP-40 homologue, subfamily B, number 6 |
| DES | Desmin |
| TNPO3 | Transportin 3 |
| HNRNPDL | Heterogeneous nuclear ribonucleoprotein D-like |
| CAPN3 | Calpain 3 |
| DYSF | Dysferlin |
| SGCG | Gamma sarcoglycan |
| SGCA | Alpha sarcoglycan |
| SGCB | Beta sarcoglycan |
| SGCD | Delta-sarcoglycan |
| TCAP | Telethonin |
| TRIM32 | Tripartite motif-containing 32 |
| FKRP | Fukutin-related protein |
| TTN | Titin |
| POMT1 | Protein-O-mannosyltransferase 1 |
| ANO5 | Anoctamin 5 |
| FKTN | Fukutin |
| POMT2 | Protein-O-mannosyltransferase 2 |
| POMGNT1 | O-linked mannose beta1,2-N-acetylglucosaminyltransferase |
| PLEC | Plectin |
| TRAPPC11 | trafficking protein particle complex 11 |
| GMPPB | GDP-mannose pyrophosphorylase B |
| DAG1 | Dystroglycan1 |
| DPM3 | Dolichyl-phosphate mannosyltransferase polypeptide 3 |
| ISPD | Isoprenoid synthase domain containing |
| VCP | Valosin-containing protein |
| LIMS2 | LIM and senescent cell antigen-like domains 2 |
| GAA | Glucosidase alpha, acid |

Congenital Muscular Dystrophies

| Gene | Protein |
|---|---|
| LAMA2 | Laminin alpha 2 chain of merosin |
| COL6A1 | Alpha 1 type VI collagen |
| COL6A2 | Alpha 2 type VI collagen |
| COL6A3 | Alpha 3 type VI collagen |
| SEPN1 | Selenoprotein N1 |
| FHL1 | Four and a half LIM domain 1 |
| ITGA7 | Integrin alpha 7 precursor |
| DNM2 | Dynamin 2 |
| TCAP | Telethonin |
| LMNA | Lamin A/C |
| FKTN | Fukutin |
| POMT1 | Protein-O-mannosyltransferase 1 |
| POMT2 | Protein-O-mannosyltransferase 2 |
| FKRP | Fukutin-related protein |
| POMGNT1 | O-linked mannose beta1,2-N-acetylglucosaminyltransferase |
| ISPD | Isoprenoid synthase domain containing |
| POMGNT2 | protein O-linked mannose N-acetylglucosaminyltransferase |
| | 2 |
| B3GNT1 | UDP-GlcNAc:betaGal beta-1,3-N-acetylglucosaminyl- |
| | transferase 1 |
| GMPPB | GDP-mannose pyrophosphorylase B |
| LARGE | Like-glycosyltransferase |
| DPM1 | Dolichyl-phosphate mannosyltransferase 1, catalytic subunit |
| DPM2 | Dolichyl-phosphate mannosyltransferase polypeptide 2, |
| | regulatory subunit |
| ALG13 | UDP-N-acetylglucosami-nyltransferase |
| B3GALNT2 | Beta-1,3-N-acetylgalacto-saminyltransferase 2 |
| TMEM5 | Transmembrane protein 5 |
| POMK | Protein-O-mannose kinase |
| CHKB | Choline kinase beta |
| ACTA1 | Alpha actin, skeletal muscle |
| TRAPPC11 | trafficking protein particle complex 11 |

Congenital Myopathies

| Gene | Protein |
|---|---|
| TPM3 | Tropomyosin 3 |
| NEB | Nebulin |
| ACTA1 | Alpha actin, skeletal muscle |
| TPM2 | Tropomyosin 2 (beta) |
| TNNT1 | Slow troponin T |
| KBTBD13 | Kelch repeat and BTB (POZ) domain containing 13 |
| CFL2 | Cofilin 2 (muscle) |
| KLHL40 | Kelch-like family member 40 |
| KLHL41 | Kelch-like family member 41 |
| LMOD3 | Leiomodin 3 (fetal) |
| SEPN1 | Selenoprotein N1 |
| RYR1 | Ryanodine receptor 1 (skeletal) |
| MYH7 | Myosin, heavy polypeptide 7, cardiac muscle, beta |
| MTM1 | Myotubularin |
| DNM2 | Dynamin 2 |
| BIN1 | Amphiphysin |
| TTN | Titin |
| SPEG | SPEG complex locus |
| MEGF10 | Multiple EGF-like-domains 10 |
| MYH2 | Myosin, heavy polypeptide 2, skeletal muscle |
| MYBPC3 | Cardiac myosin binding protein-C |
| CNTN1 | Contactin-1 |
| TRIM32 | Tripartite motif-containing 32 |
| PTPLA | Protein tyrosine phosphatase-like (3-Hydroxyacyl-CoA |
| | dehydratase |
| CACNA1S | Calcium channel, voltage-dependent, L type, alpha 1S |
| | subunit |

Distal Myopathies

| Gene | protein |
|---|---|
| DYSF | Dysferlin |
| TTN | Titin |
| GNE | UDP-N-acetylglucosamine-2-epimerase/N-acetylmannosamine |
| | kinase |
| MYH7 | Myosin, heavy polypeptide 7, cardiac muscle, beta |
| MATR3 | Matrin 3 |
| TIA1 | Cytotoxic granuleassociated RNA binding protein |
| MYOT | Myotilin |
| NEB | Nebulin |
| CAV3 | Caveolin 3 |
| LDB3 | LIM domain binding 3 |
| ANO5 | Anoctamin 5 |
| DNM2 | Dynamin 2 |
| KLHL9 | Kelch-like homologue 9 |
| FLNC | Filamin C, gamma (actin-binding protein - 280) |
| VCP | Valosin-containing protein |

Other Myopathies

| Gene | protein |
|---|---|
| ISCU | Iron-sulfur cluster scaffold homolog (E. coli) |
| MSTN | Myostatin |
| FHL1 | Four and a half LIM domain 1 |
| BAG3 | BCL2-associated athanogene 3 |
| ACVR1 | Activin A receptor, type II-like kinase 2 |
| MYOT | Myotilin |
| FLNC | Filamin C, gamma (actin-binding protein - 280) |
| LDB3 | LIM domain binding 3 |
| LAMP2 | Lysosomal-associated membrane protein 2 precursor |
| VCP | Valosin-containing protein |
| CAV3 | Caveolin 3 |
| SEPN1 | Selenoprotein N1 |
| CRYAB | Crystallin, alpha B |
| DES | Desmin |
| VMA21 | VMA21 Vacuolar H+-ATPase Homolog (S. Cerevisiae) |
| PLEC | plectin |
| PABPN1 | Poly(A) binding protein, nuclear 1 |
| TTN | Titin |
| RYR1 | Ryanodine receptor 1 (skeletal) |
| CLN3 | Ceroid-lipofuscinosis, neuronal 3 (=battenin) |
| TRIM54 | |
| TRIM63 | Tripartite motif containing 63, E3 ubiquitin protein ligase |

Myotonic Syndromes

| Gene | protein |
|---|---|
| DMPK | Myotonic dystrophy protein kinase |
| CNPB | Cellular nucleic acid-binding protein |
| CLCN1 | Chloride channel 1, skeletal muscle (Thomsen disease, |
| | autosomal dominant) |
| CAV3 | Caveolin 3 |
| HSPG2 | Perlecan |
| ATP2A1 | ATPase, Ca++ transporting, fast twitch 1 |

Ion Channel Muscle Diseases

| Gene | protein |
|---|---|
| CLCN1 | Chloride channel 1, skeletal muscle (Thomsen disease, |
| | autosomal dominant) |
| SCN4A | Sodium channel, voltage-gated, type IV, alpha |
| SCN5A | Voltage-gated sodium channel type V alpha |
| CACNA1S | Calcium channel, voltage-dependent, L type, alpha 1S |
| | subunit |
| CACNA1A | Calcium channel, voltage-dependent, P/Q type, alpha 1A |
| | subunit |
| KCNE3 | Potassium voltage-gated channel, Isk-related family, |
| | member 3 |
| KCNA1 | Potassium voltage-gated channel, shaker-related subfamily, |
| | member 1 |
| KCNJ18 | Kir2.6 (inwardly rectifying potassium channel 2.6) |
| KCNJ2 | Potassium inwardly-rectifying channel J2 |
| KCNH2 | Voltage-gated potassium channel, subfamily H, member 2 |
| KCNQ1 | Potassium voltage-gated channel, KQT-like subfamily, |
| | member 1 |
| KCNE2 | Potassium voltage-gated channel, Isk-related family, |
| | member 2 |
| KCNE1 | Potassium voltage-gated channel, Isk-related family, |
| | member 1 |

Malignant Hyperthermia

| Gene | protein |
|---|---|
| RYR1 | Ryanodine receptor 1 (skeletal) |
| CACNA1S | Calcium channel, voltage-dependent, L type, alpha |
| | 1S subunit |

Metabolic Myopathies

| Gene | protein |
|---|---|
| GAA | Acid alpha-glucosidase preproprotein |
| AGL | Amylo-1,6-glucosidase, 4-alpha-glucanotransferase |
| GBE1 | Glucan (1,4-alpha-), branching enzyme 1 (glycogen |
| | branching enzyme, Andersen disease, glycogen storage |
| | disease type IV) |
| PYGM | Glycogen phosphorylase |
| PFKM | Phosphofructokinase, muscle |
| PHKA1 | Phosphorylase b kinase, alpha submit |
| PGM1 | Phosphoglucomutase 1 |
| GYG1 | Glycogenin 1 |
| GYS1 | Glycogen synthase 3 glycogen synthase 1 (muscle) |
| | glycogen synthase 1 (muscle) |
| PRKAG2 | Protein kinase, AMP-activated, gamma 2 non-catalytic |
| | subunit |
| RBCK1 | RanBP-type and C3HC4-type zinc finger containing 1 |
| | (heme-oxidized IRP2 ubiquitin ligase 1) |
| PGK1 | Phosphoglycerate kinase 1 |
| PGAM2 | Phosphoglycerate mutase 2 (muscle) |
| LDHA | Lactate dehydrogenase A |
| ENO3 | Enolase 3, beta muscle specific |
| CPT2 | Carnitine palmitoyltransferase II |
| SLC22A5 | Solute carrier family 22 member 5 |
| SLC25A20 | Carnitine-acylcarnitine translocase |
| ETFA | Electron-transfer-flavoprotein, alpha polypeptide |
| ETFB | Electron-transfer-flavoprotein, beta polypeptide |
| ETFDH | Electron-transferring-flavoprotein dehydrogenase |
| ACADVL | Acyl-Coenzyme A dehydrogenase, very long chain |
| ABHD5 | Abhydrolase domain containing 5 |
| PNPLA2 | Adipose triglyceride lipase (desnutrin) |
| LPIN1 | Lipin 1 (phosphatidic acid phosphatase 1) |
| PNPLA8 | Patatin-like phospholipase domain containing 8 |

Other Neuromuscular Disorders

| Gene | protein |
|---|---|
| TOR1A | Torsin A |
| SGCE | Sarcoglycan, epsilon |
| IKBKAP | Inhibitor of kappa light polypeptide gene enhancer in B- |
| | cells, kinase complex-associated protein |
| TTR | Transthyretin (prealbumin, amyloidosis type I) |
| KIF21A | Kinesin family member 21A |
| PHOX2A | Paired-like aristaless homeobox protein 2A |
| TUBB3 | Tubulin, beta 3 |
| TPM2 | Tropomyosin 2 (beta) |
| MYH3 | Myosine, heavy chain 3, skeletal muscle, embryonic |
| TNNI2 | Troponin I, type 2 |
| TNNT3 | Troponin T3, skeletal |
| SYNE1 | Spectrin repeat containing, nuclear envelope 1 (nesprin 1) |
| MYH8 | Myosin heavy chain, 8, skeletal muscle, perinatal |
| POLG | Polymerase (DNA directed), gamma |
| SLC25A4 | Mitochondrial carrier; adenine nucleotide translocator |
| C10orf2 | chromosome 10 open reading frame 2 |
| POLG2 | Mitochondrial DNA polymerase, accessory subunit |
| RRM2B | Ribonucleotide reductase M2 B (TP53 inducible) |
| TK2 | Thymidine kinase 2, mitochondrial |
| SUCLA2 | Succinate-CoA ligase, ADP-forming, beta subunit |
| OPA1 | optic atrophy 1 |
| STIM1 | Stromal interaction molecule 1 |
| ORAI1 | ORAI calcium release-activated calcium modulator 1 |
| PUS1 | Pseudouridylate synthase 1 |
| CHCHD10 | Coiled-coil-helix-coiled-coil-helix domain containing 10 |
| CASQ1 | Calsequestrin 1 (fast-twitch, skeletal muscle) |
| YARS2 | tyrosyl-tRNA synthetase 2, mitochondrial |

In some embodiments, the target gene for gene therapy (additive gene therapy or gene editing) is a gene responsible for one of the muscular dystrophies listed above, in particular DMD or BMD (DMD gene); LGMDs (DNAJB6, FKRP CAPN3, DYSF, SGCG, SGCA, SGCB, SGCD, ANO5 genes and others).

According to specific embodiments and in relation to muscular dystrophy, the transgene may encode a protein selected in the group consisting of: dystrophin (including microdystrophin, minidystrophin, quasidystrophin), HSP-40 homologue B6, calpain 3, dysferlin (DYSF), sarcoglycan (α, β, γ, δ), FKRP (Fukutin-Related Protein) and Anoctamin5.

According to a more specific embodiment, the gene of interest encodes a calpain 3 protein, advantageously the human calpain 3, more advantageously of sequence SEQ ID NO: 8.

The sequence encoding said protein, also named ORF for “open reading frame”, is a nucleic acid sequence or a polynucleotide and may in particular be a single- or double-stranded DNA (deoxyribonucleic acid), an RNA (ribonucleic acid) or a cDNA (complementary deoxyribonucleic acid).

Advantageously, said sequence or transgene encodes a functional protein, i.e. a protein capable of ensuring its native or essential functions, especially in the skeletal muscles. This implies that the protein produced using the expression system of the invention is properly expressed and located, and is active.

According to a preferred embodiment, said sequence encodes the native protein, said protein being preferably of human origin. It may also be a derivative or a fragment of this protein, provided that the derivative or fragment retains the desired activity. Preferably, the term “derivative” or “fragment” refers to a protein sequence having at least 50%, preferably 60%, even more preferably 70% or even 80%, 85%, 90%, 95%, 96%, 97%, 98% or 99% identity with the native human sequence. Proteins from another origin (non-human mammals, etc.) or truncated, or even mutated, but active proteins are for instance encompassed. Thus and in the context of the invention, the term “protein” is understood as the full-length protein regardless of its origin, as well as functional derivatives and fragments thereof.

As already mentioned and according to one specific embodiment, the promoter according to the invention, having high activity in the skeletal muscles and low activity in the heart, has no activity or a low activity in non-target tissues, e.g. in the liver, the brain or the kidneys . . . as mentioned above. Alternatively, the expression system according to the invention further comprises a sequence which allows controlling the expression of the therapeutic transgene of interest by preventing, decreasing or suppressing its expression in non-target tissues or in tissues wherein the encoded protein can be toxic, e.g. the heart, by stabilizing the mRNA coding for the protein of interest, such as a therapeutic protein, encoded by the gene of interest. These sequences include, for example, silencers (such as tissue-specific silencers), microRNA target sequences, introns and polyadenylation signals.

In the context of the invention, the terminology “prevent the expression” preferably refers to cases where, even in the absence of the said sequence, there is no expression, while the terminology “decrease the level of expression” refers to cases where the expression is decreased (or reduced) by the provision of said sequence.

Advantageously, said sequence is capable of preventing the expression or reducing the level of expression of the transgene in the tissues wherein protein expression is not of interest or may be toxic. This action may take place according to various mechanisms, particularly:
with regard to the level of transcription of the sequence encoding the protein;
with regard to transcripts resulting from the transcription of the sequence encoding the protein, e.g., via their degradation;
with regard to the translation of the transcripts into protein.

Such a sequence is preferably a target for a small RNA molecule e.g. selected from the following group:
microRNAs;
endogenous small interfering RNA or siRNAs;
small fragments of the transfer RNA (tRNA);
RNA of the intergenic regions;
Ribosomal RNA (rRNA);
Small nuclear RNA (snRNA);
Small nucleolar RNAs (snoRNA);
RNA interacting with piwi proteins (piRNA).

Advantageously, this sequence has no negative impact on the transgene expression in the target tissue(s), especially in the skeletal muscles.

Preferably, such a sequence is selected for its effectiveness in the tissue wherein the expression of the protein has no therapeutic activity or is toxic. Since the effectiveness of this sequence can be variable depending on the tissues, it may be necessary to combine several of these sequences, chosen for their effectiveness in said tissues.

According to a preferred embodiment, this sequence is a target sequence for a microRNA (miRNA). As known, such a judiciously chosen sequence helps to specifically suppress gene expression in selected tissues.

Thus and according to a particular embodiment, the expression system of the invention further comprises a target sequence for a microRNA (miRNA) expressed or present in the tissue(s) in which the expression of the protein has no therapeutic activity and/or is toxic. Suitably, the quantity of this miRNA present in the target tissue, especially the skeletal muscles, is less than that present in the tissues wherein the transgene is useless or even toxic, or this miRNA may not even be expressed in the target tissues. According to a particular embodiment, the target miRNA is not expressed in the skeletal muscles and possibly in the heart.

As is known to the person skilled in the art, the presence or level of expression, particularly in a given tissue, of a miRNA may be assessed by PCR, preferably by RT-PCR, or by Northern blot.

Different miRNAs, as well as their target sequence and their tissue specificity, are known to those skilled in the art and are for example described in the document WO 2007/000668. MiRNAs expressed in the liver are e.g. miR-122.

According to a particular embodiment and in case no expression of the transgene is desired in the heart, the expression system according to the invention can comprise one or more copies of a target sequence for a miRNA expressed in the heart, e.g. for miR208a. According to a specific embodiment, such a target sequence can also be used in tandem.

As an example, a possible target sequence for miR208a corresponds to nucleotides 3411 to 3432 or 3439 to 3460 of SEQ ID NO: 7. However, any derivatives thereof able to bind miR208a can be used.

According to the invention, an expression system comprises the elements necessary for the expression of the transgene present. In addition to sequences such as those defined above to ensure and to modulate transgene expression, such a system may include other sequences such as:
a sequence for transcript stabilization, e.g. intron 2/exon 3 (modified) of the gene coding the human β globin (HBB2). Said intron is advantageously followed by consensus Kozak sequence (GCCACC) included before AUG start codon within mRNA, to improve initiation of translation;
a polyadenylation signal, e.g. the polyA of the gene of interest, the polyA of SV40 or of beta hemoglobin (HBB2), advantageously in 3′ of the transgene;
enhancer sequences.

An expression system according to the invention can be introduced in a cell, a tissue or a body, particularly in humans. In a manner known to those skilled in the art, the introduction can be done ex vivo or in vivo, for example by transfection or transduction. According to another aspect, the present invention therefore encompasses a cell or a tissue, preferably of human origin, comprising an expression system of the invention. Such a, expression system or cells can be used for the in vitro production of the encoded protein.

The expression system according to the invention, i.e. an isolated nucleic acid, can be administered in a subject, namely in the form of a naked DNA. To facilitate the introduction of this nucleic acid in the cells, it can be combined with different chemical means such as colloidal disperse systems (macromolecular complex, nanocapsules, microspheres, beads) or lipid-based systems (oil-in-water emulsions, micelles, liposomes).

Alternatively and according to a preferred embodiment, the expression system of the invention comprises a plasmid or a vector. Advantageously, such a vector is a viral vector. Viral vectors commonly used in gene therapy in mammals, including humans, are known to those skilled in the art. Such viral vectors are preferably chosen from the following list: vector derived from the herpes virus, baculovirus vector, lentiviral vector, retroviral vector, adenoviral vector and adeno-associated viral vector (AAV).

According to a specific embodiment of the invention, the viral vector containing the expression system is an adeno-associated viral (AAV) vector.

Adeno-associated viral (AAV) vectors have become powerful gene delivery tools for the treatment of various disorders. AAV vectors possess a number of features that render them ideally suited for gene therapy, including a lack of pathogenicity, moderate immunogenicity, and the ability to transduce post-mitotic cells and tissues in a stable and efficient manner. Expression of a particular gene contained within an AAV vector can be specifically targeted to one or more types of cells by choosing the appropriate combination of AAV serotype, promoter, and delivery method.

In one embodiment, the encoding sequence is contained within an AAV vector. More than 100 naturally occurring serotypes of AAV are known. Many natural variants in the AAV capsid exist, allowing identification and use of an AAV with properties specifically suited for dystrophic pathologies. AAV viruses may be engineered using conventional molecular biology techniques, making it possible to optimize these particles for cell specific delivery of nucleic acid sequences, for minimizing immunogenicity, for tuning stability and particle lifetime, for efficient degradation, for accurate delivery to the nucleus.

As mentioned above, the use of AAV vectors is a common mode of exogenous delivery of DNA as it is relatively non-toxic, provides efficient gene transfer, and can be easily optimized for specific purposes. Among the serotypes of AAVs isolated from human or non-human primates (NHP) and well characterized, human serotype 2 is the first AAV that was developed as a gene transfer vector. Other currently used AAV serotypes include AAV1, AAV3, AAV4, AAV5, AAV6, AAV7, AAV8, AAV9, AAVrh10, AAVrh74, AAV11, AAV12 and their variants. In addition, non-natural engineered variants and chimeric AAV can also be useful.

Desirable AAV fragments for assembly into vectors include the cap proteins, including the vp1, vp2, vp3 and hypervariable regions, the rep proteins, including rep 78, rep 68, rep 52, and rep 40, and the sequences encoding these proteins. These fragments may be readily utilized in a variety of vector systems and host cells.

Such fragments may be used alone, in combination with other AAV serotype sequences or fragments, or in combination with elements from other AAV or non-AAV viral sequences. As used herein, artificial AAV serotypes include, without limitation, AAV with a non-naturally occurring capsid protein. Such an artificial capsid may be generated by any suitable technique, using a selected AAV sequence (e.g., a fragment of a vp1 capsid protein) in combination with heterologous sequences which may be obtained from a different selected AAV serotype, non-contiguous portions of the same AAV serotype, from a non-AAV viral source, or from a non-viral source (i.e. its capsid comprises VP capsid proteins derived from at least two different AAV serotypes, or comprises at least one chimeric VP protein combining VP protein regions or domains derived from at least two AAV serotypes). An artificial AAV serotype may be, without limitation, a chimeric AAV capsid, a recombinant AAV capsid, or a “humanized” AAV capsid. Moreover, a peptide (P) can be introduced in said capsids, for example into a variable region of the cap gene, possibly to modify the AAV tropism.

In one embodiment, the vectors useful in the compositions and methods described herein contain, at a minimum, sequences encoding a selected AAV serotype capsid, e.g., an AAV8 capsid, or a fragment thereof. In another embodiment, useful vectors contain, at a minimum, sequences encoding a selected AAV serotype rep protein, e.g., AAV8 rep protein, or a fragment thereof. Optionally, such vectors may contain both AAV cap and rep proteins. In vectors in which both AAV rep and cap are provided, the AAV rep and AAV cap sequences can both be of one serotype origin, e.g., all AAV8 origin. Alternatively, vectors may be used in which the rep sequences are from an AAV serotype, which differs from that which is providing the cap sequences. In one embodiment, the rep and cap sequences are expressed from separate sources (e.g., separate vectors, or a host cell and a vector). In another embodiment, these rep sequences are fused in frame to cap sequences of a different AAV serotype to form a chimeric AAV vector. In some embodiments the AAV vector comprises a genome and a capsid derived from AAVs of different serotypes.

Thus exemplary AAVs, or artificial AAVs, include AAV2/8 (U.S. Pat. No. 7,282,199), AAV2/5 (available from the National Institutes of Health), AAV2/9 (WO2005/033321), AAV2/6 (U.S. Pat. No. 6,156,303), AAVrh10 (WO2003/042397), AAVrh74 (WO2003/123503), AAV9-rh74 hybrid or AAV9-rh74-P1 hybrid (WO2019/193119; WO2020/200499; EP20306005.8).

According to one embodiment, the AAV is of serotype 2, 5, 8 or 9, or an AAVrh74. Advantageously, the claimed vector comprises a capsid selected in the group consisting of: AAV8 capsid, AAV9 capsid, AAV9-rh74 capsid and AAV9-rh74-P1 capsid.

In the AAV vectors used in the present invention, the AAV genome may be either a single stranded (ss) nucleic acid or a double stranded (ds)/self complementary (sc) nucleic acid molecule.

Advantageously, the gene of interest or transgene is inserted between the ITR («Inverted Terminal Repeat») sequences of the AAV vector. Typically, ITR sequences originate from AAV2 or AAV9, advantageously AAV2.

Recombinant viral particles can be obtained by any method known to the one skilled in the art, e.g. by co-transfection of 293 HEK cells, by the herpes simplex virus system and by the baculovirus system. The vector titers are usually expressed as viral genomes per mL (vg/mL).

In one embodiment, the vector comprises regulatory sequences including a promoter according to the invention as described above.

In relation to a polynucleotide encoding the sequence SEQ ID NO: 8, a vector of the invention may comprise the sequence shown in sequence SEQ ID NO: 7.

According to a specific embodiment, the expression system of the invention corresponds to an AAV9-rh74-P1 hybrid, advantageously as disclosed in EP20306005.8, harboring a sequence containing:
a promoter according to the invention, advantageously of sequence SEQ ID NO: 2; or a sequence having identity greater than or equal to 90% with SEQ ID NO: 2;
a sequence encoding calpain 3, advantageously of sequence SEQ ID NO: 8, placed under the control of said promoter;
at least one target sequence of mir208a, possibly two target sequences in tandem, located in 3′ of the sequence encoding calpain 3.

According to another specific embodiment, the expression system of the invention corresponds to an AAV9-rh74-P1 hybrid, advantageously as disclosed in EP20306005.8, harboring a sequence containing:
a promoter according to the invention, advantageously of sequence SEQ ID NO: 2; or a sequence having identity greater than or equal to 90% with SEQ ID NO: 2;
a sequence encoding calpain 3, advantageously of sequence SEQ ID NO: 8, placed under the control of said promoter;
one target sequence of mir208a, located in 3′ of the sequence encoding calpain 3.

According to a preferred embodiment, the expression system of the invention includes a vector having a suitable tropism, in this case higher for the target tissue(s), advantageously the skeletal muscles and the heart, than for the tissues where the expression of the protein is not desired.

Further aspects of the invention concern:
A cell comprising the expression system of the invention or a vector comprising said expression system, as disclosed above.

The cell can be any type of cells, i.e. prokaryotic or eukaryotic. The cell can be used for propagation of the vector or can be further introduced (e.g. grafted) in a host or a subject. The expression system or vector can be introduced in the cell by any means known in the art, e.g. by transformation, electroporation or transfection. Vesicles derived from cells can also be used.
A transgenic animal, advantageously non-human, comprising the expression system of the invention, a vector comprising said expression system, or a cells comprising said expression system or said vector, as disclosed above.

Another aspect of the invention relates to a composition comprising an expression system, a vector or a cell, as disclosed above, for use as a medicament.

According to an embodiment, the composition comprises at least said gene therapy product (the expression system, the vector or the cell), and possibly other active molecules (other gene therapy products, chemical molecules, peptides, proteins . . . ), dedicated to the treatment of the same disease or another disease.

According to a specific embodiment, the use of the expression system according to the invention is combined with the use of anti-inflammatory drugs such as corticoids.

The present invention then provides pharmaceutical compositions comprising an expression system, a vector or a cell of the invention. Such compositions comprise a therapeutically effective amount of the therapeutic (the expression system or vector or cell of the invention), and a pharmaceutically acceptable carrier. In a specific embodiment, the term “pharmaceutically acceptable” means approved by a regulatory agency of the Federal or a state government or listed in the U.S. or European Pharmacopeia or other generally recognized pharmacopeia for use in animals, and humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, sodium stearate, glycerol monostearate, talc, sodium chloride, dried skim milk, glycerol, propylene glycol, water, ethanol and the like.

The composition, if desired, can also contain minor amounts of wetting or emulsifying agents, or pH buffering agents. These compositions can take the form of solutions, suspensions, emulsions, sustained-release formulations and the like. Examples of suitable pharmaceutical carriers are described in “Remington's Pharmaceutical Sciences” by E. W. Martin. Such compositions will contain a therapeutically effective amount of the therapeutic, preferably in purified form, together with a suitable amount of carrier so as to provide the form for proper administration to the subject.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lidocaine to release pain at the site of the injection.

In one embodiment, the composition according to the invention is suitable for administration in humans. The composition is preferably in a liquid form, advantageously a saline composition, more advantageously a phosphate buffered saline (PBS) composition or a Ringer-Lactate solution.

The amount of the therapeutic (i.e. an expression system or a vector or a cell) of the invention which will be effective in the treatment of the target diseases can be determined by standard clinical techniques. In addition, in vivo and/or in vitro assays may optionally be employed to help predict optimal dosage ranges. The precise dose to be employed in the formulation will also depend on the route of administration, the physical characteristics of the individual under consideration such as sex, age and weight, concurrent medication, other factors and the seriousness of the disease, and should be decided according to the judgment of the practitioner and each patient's circumstances.

Suitable administration should allow the delivery of a therapeutically effective amount of the gene therapy product to the target tissues, especially skeletal muscles and possibly heart. In the context of the invention, when the gene therapy product is a viral vector, the therapeutic dose is defined as the quantity of viral particles (vg for viral genomes) containing the transgene administered per kilogram (kg) of the subject.

In case of a treatment comprising administering a viral vector, such as an AAV vector, to the subject, typical doses of the vector are of at least 1×10⁸ vector genomes per kilogram body weight (vg/kg), such as at least 1×10⁹ vg/kg, at least 1×10¹⁰ vg/kg, at least 1×10¹¹ vg/kg, at least 1×10¹² vg/kg at least 1×10¹³ vg/kg, at least 1×10¹⁴ vg/kg, at least 1015 vg/kg.

Specifically, the dose can be between 5·10¹¹ vg/kg and 10¹⁴ vg/kg, e.g. 1, 2, 3, 4, 5, 6, 7, 8 or 9·10¹³ vg/kg. A lower dose of e.g. 1, 2, 3, 4, 5, 6, 7, 8 or 9·10¹² vg/kg can also be contemplated in order to avoid potential toxicity and/or immune reactions. As known by the skilled person, a dose as low as possible giving a satisfying result in term of efficiency is preferred.

Available routes of administration are topical (local), enteral (system-wide effect, but delivered through the gastrointestinal (GI) tract), or parenteral (systemic action, but delivered by routes other than the GI tract). The preferred route of administration of the compositions disclosed herein is parenteral which includes intramuscular administration (i.e. into the muscle) and systemic administration (i.e. into the circulating system). In this context, the term “injection” (or “perfusion” or “infusion”) encompasses intravascular, in particular intravenous (IV), intramuscular (IM), intraocular, intrathecal or intracerebral administration. Injections are usually performed using syringes or catheters.

In one embodiment, systemic delivery of the composition comprises administering the composition near a local treatment site, i.e. in a vein or artery nearby a weakened muscle. In certain embodiments, the invention comprises the local delivery of the composition, which produces systemic effects. This route of administration, usually called “regional (loco-regional) infusion”, “administration by isolated limb perfusion” or “high-pressure transvenous limb perfusion” has been successfully used as a gene delivery method in muscular dystrophy.

According to one aspect, the composition is administered to an isolated limb (loco-regional) by infusion or perfusion. In other words, the invention comprises the regional delivery of the composition in a leg and/or arm by an intravascular route of administration, i.e. a vein (transvenous) or an artery, under pressure. This is usually achieved by using a tourniquet to temporarily arrest blood circulation while allowing a regional diffusion of the infused product, as e.g. disclosed by Toromanoff et al. (2008).

In one embodiment, the composition is injected in a limb of the subject. When the subject is a human, the limb can be the arm or the leg. According to one embodiment, the composition is administered in the lower part of the body of the subject, e.g. below the knee, or in the upper part of the body of the subject, e.g., below the elbow.

A preferred method of administration according to the invention is systemic administration. Systemic injection opens the way to an injection of the whole body, in order to reach the entire muscles of the body of the subject including the heart and the diaphragm and then a real treatment of these systemic and still incurable diseases. In certain embodiments, systemic delivery comprises delivery of the composition to the subject such that composition is accessible throughout the body of the subject.

According to a preferred embodiment, systemic administration occurs via injection of the composition in a blood vessel, i.e. intravascular (intravenous or intra-arterial) administration. According to one embodiment, the composition is administered by intravenous injection, through a peripheral vein.

In a specific embodiment, the treatment comprises a single administration of the composition.

Such compositions are notably intended for gene therapy in a subject, particularly for the treatment of diseases due the deficiency of the above-identified proteins, especially neuromuscular disease and muscular dystrophy. Thus, by gene editing or gene replacement, a correct version of the gene is provided in muscle cells of affected patients and this may contribute to effective therapies against the diseases as listed below.

In some embodiments, the pharmaceutical composition of the invention is for use for treating muscular diseases (i.e., myopathies) or muscular injuries, in particular neuromuscular genetic disorders, with no liver damage, such as for example: muscular dystrophies, congenital muscular dystrophies, congenital myopathies, distal myopathies, other myopathies, myotonic syndromes, ion channel muscle diseases, malignant hyperthermia, metabolic myopathies, and other neuromuscular disorders, advantageously muscular dystrophies, congenital muscular dystrophies, congenital myopathies, distal myopathies and other myopathies.

Muscular dystrophies include in particular:
Dystrophinopathies, a spectrum of X-linked muscle diseases caused by pathogenic variants in DMD gene, which encodes the protein dystrophin. Dystrophinopathies comprise Duchenne muscular dystrophy (DMD), Becker muscular dystrophy (BMD) and DMD-associated dilated cardiomyopathy;
The Limb-girdle muscular dystrophies (LGMDs) which are a group of disorders that are clinically similar to DMD but occur in both sexes as a result of autosomal recessive and autosomal dominant inheritance. Limb-girdle dystrophies are caused by mutation of genes that encode sarcoglycans and other proteins associated with the muscle cell membrane, which interact with dystrophin. The term LGMD1 refers to genetic types showing dominant inheritance (autosomal dominant), whereas LGMD2 refers to types with autosomal recessive inheritance. Pathogenic variants at more than 50 loci have been reported (LGMD1A to LGMD1H; LGMD2A to LGMD2Y).
Calpainopathy (LGMD2A) is caused by mutation of the gene CAPN3 with more than 450 pathogenic variants described.

A non-limiting list of such diseases includes: centronuclear myopathy, advantageously X-linked myotubular myopathy (XLMTM) and Charcot-Marie-Tooth disease, limb-girdle muscular dystrophy, advantageously LGMD2A, LGMD2B, LGMD2D or LGMD2I, LGMD1D, LGMD2L Congenital Muscular Dystrophy type 1C (MDC1C), Walker-Warburg Syndrome (WWS), Muscle-Eye-Brain disease (MEB), Duchenne (DMD) or Becker (BMD) muscular dystrophy, congenital muscular dystrophy with selenoprotein N deficiency, congenital muscular dystrophy with primary merosin deficiency, Ullrich congenital muscular dystrophy, central core congenital myopathy, multi-minicore congenital myopathy, centronuclear autosomal myopathy, myopathy with fibre dysproportion, nemaline myopathy, congenital myasthenic syndromes, miyoshi distal myopathy, dysferlinopathies, dystroglycanopathies and sarcoglycanopathies. In some embodiments the pharmaceutical composition of the invention is for use for treating Duchenne (DMD) or Becker (BMD) muscular dystrophy, congenital muscular dystrophy limb-girdle muscular dystrophy, advantageously LGMD2A, LGMD2B, LGMD2D, LGMD2I, LGMD1D or LGMD2L.

A specific example of gene editing would be the treatment of Limb-girdle muscular dystrophy 2A (LGMD2A) which is caused by mutations in the calpain-3 gene (CAPN3). Other examples would be the treatment of mutations in the DMD gene.

Subjects that could benefit from the compositions of the invention include all patients diagnosed with such a disease or at risk of developing such a disease. A subject to be treated can then be selected based on the identification of mutations or deletions in the gene encoding the above-listed proteins by any method known to the one skilled in the art, including for example sequencing of said gene, and/or through the evaluation of the protein level of expression or activity by any method known to the one skilled in the art. Therefore, said subjects include both subjects already exhibiting symptoms of such a disease and subjects at risk of developing said disease. In one embodiment, said subjects include subjects already exhibiting symptoms of such a disease. In another embodiment, said subjects are ambulatory patients and early non-ambulant patients.

More generally and according to further embodiments, an expression system according to the invention is useful for:
increasing muscular force, muscular endurance and/or muscle mass in a subject;
reducing fibrosis in a subject;
reducing contraction-induced injury in a subject;
treating muscular dystrophy in a subject;
reducing degenerating fibers or necrotic fibers in a subject suffering from muscular dystrophy;
reducing inflammation in a subject suffering from muscular dystrophy;
reducing levels of creatine kinase (or any other dystrophic marker) in a subject suffering from muscular dystrophy;
treating myofiber atrophy and hypertrophy in a subject suffering from muscular dystrophy;
decreasing dystrophic calcification in a subject suffering from muscular dystrophy;
decreasing fatty infiltration in a subject;
decreasing central nucleation in a subject.

According to one embodiment, the present invention concerns a method for treating such conditions comprising administering to a subject the gene therapy product (expression system, vector or cell) as disclosed above.

Advantageously, the expression system is administered systemically in the body, particularly in an animal, advantageously in mammals and more preferably in humans.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, fourth edition (Sambrook, 2012); “Oligonucleotide Synthesis” (Gait, 1984); “Culture of Animal Cells” (Freshney, 2010); “Methods in Enzymology” “Handbook of Experimental Immunology” (Weir, 1997); “Gene Transfer Vectors for Mammalian Cells” (Miller and Calos, 1987); “Short Protocols in Molecular Biology” (Ausubel, 2002); “Polymerase Chain Reaction: Principles, Applications and Troubleshooting”, (Babar, 2011); “Current Protocols in Immunology” (Coligan, 2002). These techniques are applicable to the production of the polynucleotides and polypeptides of the invention, and, as such, may be considered in making and practicing the invention. Particularly useful techniques for particular embodiments will be discussed in the sections that follow.

The disclosures of each and every patent, patent application, and publication cited herein are hereby incorporated herein by reference in their entirety.

Without further description, it is believed that one of ordinary skill in the art can, using the preceding description and the following illustrative examples, make and utilize the compounds of the present invention and practice the claimed methods.

### EXPERIMENTAL EXAMPLES

The invention is further described in detail by reference to the following experimental examples and the attached figures. These examples are provided for purposes of illustration only, and are not intended to be limiting.

In particular, the invention is illustrated in relation to an AAV9 vector comprising a transgene placed under the control of the truncated ACTA1 promoter according to the invention (noted ACTA1) compared to the human desmin promoter.

### FIGURES

FIG. 1: Luciferase activity of GFP-Luc transgene in C57Bl6 albino mice (4 males/15 promoter) injected with 5^{e}13 vg/kg AAV9-promoter-GFP-Luc (promoter=Desmin or ACTA1)

A/ Imaging analysis of the injected mice

B/ Luciferase activity normalized by total protein amount in the different organs (TA: tibialis anterior muscle; diaphragm; heart; liver; kidney; adr glands: adrenal glands)

FIG. 2: Calpain 3 expression in the TA (tibialis anterior) muscle and in the heart of mice (2 or 3 males/promoter) not injected (NI) or injected with 1^{c}14 vg/kg AAV9-promoter-hCalpain3-2×target-miR208a

2T: 2×target-miR208a

promoter=Desmin: Des or ACTA1: ACTA

A/ at RNA level by qRT-PCR after normalization with Rplp0

B/ at protein level by western blot using an antibody directed against calpain 3.

### MATERIALS AND METHODS

Animal Models

The animal studies were performed in accordance to the current European legislation on animal care and experimentation (2010/63/EU) and approved by the institutional ethics committee of the Centre d'Exploration et de Recherche Fonctionnelle Expérimentale in Evry, France (protocol APAFIS DAP 2018-024-B#19736). The C57Bl6 albino mice were ordered to the Charles River Facility.

Expressing Cassette and AAV-Mediated Gene Transfer

Two different AAV cassettes were designed using the AAV2 ITR sequences, the fusion transgene GFP-Luciferase and the SV40 polyadenylation sequence. The promoter was the only element that differed between the constructs. In this study, the human desmin (Des) promoter (SEQ ID NO: 5) and the truncated ACTA1 promoter of the invention (SEQ ID NO: 2) were compared. The serotype 9 was used for the production of GFP-Luc recombinant adeno-associated virus (AAV9-promoter-GFP-Luc) using the tri-transfection method. The corresponding sequence including the ITR sequences is shown in SEQ ID NO: 6 in relation to the truncated ACTA1 promoter.

Two other AAV cassettes were also designed using the same promoters but with the human calpain 3 transgene and:
AAV9 ITR sequences;
HBB2 intron inserted between the promoter and the transgene;
two target sequences of mir208a in tandem inserted between the transgene and the HBB2 polyadenylation sequence.

The serotype 9 was used for the production of recombinant calpain 3 adeno-associated virus (AAV9-promoter-hCalpain3-2×target-miR208a) using the tri-transfection method. The corresponding sequence including the ITR sequences is shown in SEQ ID NO: 7 in relation to the truncated ACTA1 promoter.

The different vectors were injected by a single systemic administration in the tail vein of male one month-old C57Bl6 Albino mice or C57Bl6 mice in order to express the GFP-Luc transgene or to produce the human calpain 3, respectively. The doses of vector injected were normalized by the body's weight of mice at 5e13vg/kg of AAV9-promoter-GFP-Luc or at 1e14vg/kg of AAV9-promoter-hCalpain3-2×target-miR208a. Two weeks after treatment with AAV9-promoter-GFP-Luc, global body biodistribution was assessed by luciferase imaging in living animals. Three or four weeks after treatment with AAV9-promoter-GFP-Luc and AAV9-promoter-hCalpain3-2×target-miR208a, respectively, mice were sacrificed and tissues collected. The tibialis anterior (TA) muscle was chosen as a representative skeletal muscle.

Quantification of the luciferase activity by luciferase assay Samples were first homogenized with 500 μL of assay buffer (Tris/Phosphate, 25 mM; Glycerol 15%; DTT, 1 mM; EDTA 1 mM; MgCl2 8 mM) with 0.2% of Triton X-100 and Protease inhibitor cocktail PIC (Roche). Ten μl of lysate were loaded into flat-bottomed wells of a white opaque 96-well plate. The Enspire spectrophotometer was used for quantification of the luminescence. The pumping system delivers D-luciferin (167 μM; Interchim) and assay buffer with ATP (40 nM) (Sigma-Aldrich) to each well of the plate. The signal of Relative Light Unit (RLU) was measured after each dispatching of D-luciferin and ATP, respecting 2 sec delay between each samples. A BCA protein quantification (Thermo Scientific) was performed to normalize the quantity of protein in each sample. The result was expressed as the level of RLU normalized by the protein amount.

Global Body Biodistribution

Mice were anesthetized by inhalation of isoflurane and injected intraperitoneally with 50 mg/ml D-luciferin (LifeTechnologies, California, USA). In vivo imaging was performed using IVIS® Lumina Imaging system (PerkinElmer). The software Living Image® (PerkinElmer) was used to analyze the images.

mRNA Quantification

Total RNA extraction was performed from frozen tissues following NucleoSpin® RNA Set for NucleoZOL protocol (Macherey Nagel). Extracted RNA was eluted in 60 μl of RNase-free water and treated with Free DNA kit (Ambion) to remove residual DNA. Total RNA was quantified using a Nanodrop spectrophotometer (ND8000 Labtech).

For quantification of the transgene expression, one μg of RNA was reverse-transcribed using the RevertAid H minus Reverse transcriptase kit (Thermo Fisher Scientific) and a mixture of random oligonucleotides and oligo-dT. Real-time PCR was performed using LightCycler480 (Roche) using specific sets of primers and probes (Thermo Fisher Scientific) for the quantification of human calpain 3:

(SEQ ID NO: 9)
FWD: 5′-CGCCTCCAAGGCCCGT-3′
(SEQ ID NO: 10)
REV: 5′-GGCGGAAGCGCTGGCT-3′
and
(SEQ ID NO: 11)
Probe: 5′-CTACATCAACATGAGAGAGGT-3′.

For mouse samples, the Rplp0 was used to normalize the data across samples:

(SEQ ID NO: 12)
FWD: 5′-CTCCAAGCAGATGCAGCAGA-3′
(SEQ ID NO: 13)
REV: 5′-ATAGCCTTGCGCATCATGGT-3′,
and
(SEQ ID NO: 14)
Probe: 5′-CCGTGGTGCTGATGGGCAAGAA-3′.

Each experiment was performed in duplicate. Quantification cycle (Cq) values were calculated with the LightCycler® 480 SW 1.5.1 using 2nd Derivative Max method. RT-qPCR results, expressed as raw Cq, were normalized to Rplp0. The relative expression was calculated using the 2^{-ΔCt} method.

Western Blot Analysis

Frozen sections of approximately 1 mm of tissues (Heart and TA muscle) were solubilized in radio immunoprecipitation assay (RIPA) buffer with protease inhibitor cocktail. For calpain 3, 1 mg of tissue was mixed with 40 μl of urea buffer (8M Urea, 2M Thiourea, 3% SDS, 50 mM Tris-HCl pH 6.8, 0.03% Bromophenol Blue pH 6.8, 50% Ultra-pure Glycerol+Protease inhibitor Cocktail (100×) Sigma P8340) and 40 μl of glycerol. Protein extract was quantified by BCA (bicinchoninic acid) protein assay (Pierce). Thirty μg of total protein were processed for western blot analysis, using calpain 3 antibody (CALP-12A2 (Leica Biosystem); COP-COP-080049 (Cosmo Bio)). Fluorescence signal of the secondary antibodies was read on an Odyssey imaging system, and band intensities were measured by the Odyssey application software (LI-COR Biosciences, 2.1 version).

Statistical Analyses

Statistical analyses were performed using the GraphPad Prism version 6.04 (GraphPad Software, San Diego, Calif.). Statistical analyses were performed by ANOVA for all experiments. Data were expressed as mean±SD. P values of less than 0.05 were considered statistically significant.

Results:

I/ Expression Profile of the New Promoter in Comparison with the Desmin Promoter Using the Reporter Gene GFP-Luc:

Two weeks after injection in the tail vein of four male one month-old C57Bl6 Albino mice of 5e13vg/kg AAV9-promoter-GFP-Luc, global body biodistribution was assessed by luciferase imaging in living animals using the IVIS system.

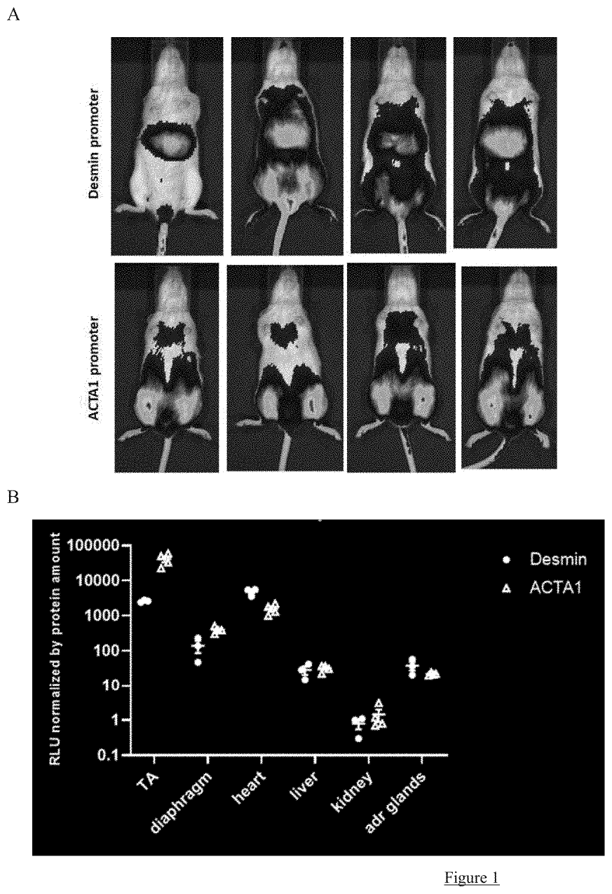

The global body biodistribution was shown to be different between the two promoters (FIG. 1A). Indeed, luciferase imaging revealed a stronger signal in limb muscles for the truncated ACTA1 promoter compared to the Desmin promoter, as well as a lower signal in the thorax.

At day 21, mice were sacrificed. It is to be noted that one mouse was found dead in the Desmin promoter group. No mortality was observed in the truncated ACTA1 promoter group.

The luciferase activity was biochemically measured in the sampled muscles and organs, then normalized to the amount of proteins in each sample. The level of luciferase activity is higher in skeletal muscles with the truncated ACTA1 promoter compared to the Desmin promoter (see TA and diaphragm), and lower in the heart (FIG. 1B). No change is observed in the other organs analysed, i.e. liver, kidney and adrenal glands.

Taken together, these results show that the use of the truncated ACTA1 promoter according to the invention leads to a higher level of transgene expression in skeletal muscles, and a lower level in cardiac muscle, compared to the use of Desmin promoter.

II/ Validation of the Expression Profile of the New Promoter in Comparison with the Desmin Promoter Using the Calpain 3 Transgene:

To validate these observations and to confirm that the truncated ACTA1 promoter is adapted for e.g. driving the expression for the human calpain 3 transgene, C57Bl6 male mice (2 or 3 per group) were intravenously injected with the rAAVs (AAV9-promoter-hCalpain3-2×target-miR208a) at the dose of 1e14 vg/kg. Four weeks after injection, the animals were euthanized. Muscles and heart were sampled for molecular analyses.

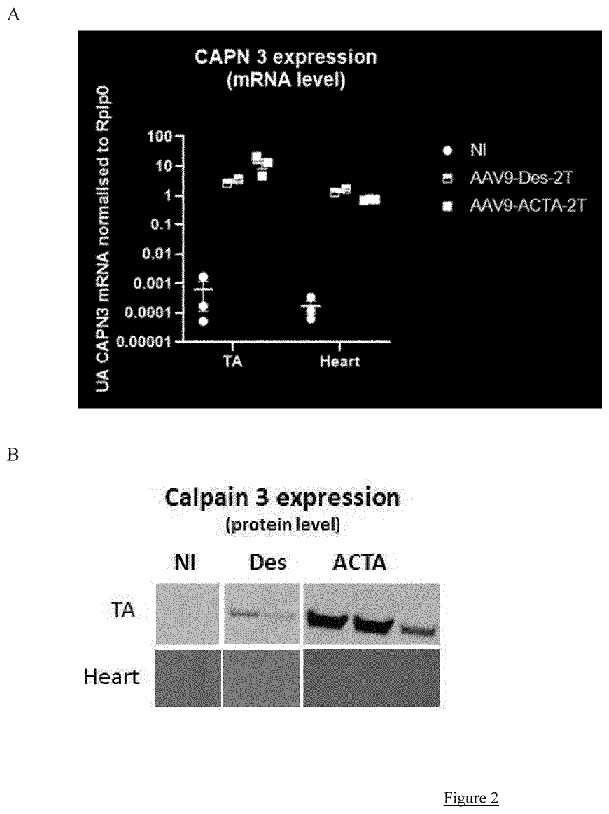

The calpain 3 expression was measured at mRNA levels (FIG. 2A). The level of Calpain 3 mRNA is higher in Tibialis anterior (TA) and lower in the heart for the truncated ACTA1 promoter compared to the Desmin promoter.

The levels of Calpain 3 protein was also measured (FIG. 2B). The level of Calpain 3 is higher in the TA muscle for the truncated ACTA1 promoter compared to the Desmin promoter. In an expected manner, there is no expression of Calpain 3 protein in the heart with both promoters, because of the presence in the cassettes of two target sequences for miR208a.

### CONCLUSIONS

All these results show that, compared to the Desmin promoter, the small-sized truncated ACTA1 promoter according to the invention leads to a higher level of transgene expression in skeletal muscles, and a lower level in heart.

1. A promoter comprising:
the sequence SEQ ID NO: 2; or
a sequence having identity greater than or equal to 90% with SEQ ID NO: 2.

2. The promoter according to claim 1, wherein its size does not exceed 350 nucleotides.

3. The promoter according to claim 1, wherein it ensures an expression level in the skeletal muscles higher than in the heart.

4. The promoter according to claim 1, wherein it comprises or consists of the sequence SEQ ID NO: 2.

5. An expression system comprising a promoter comprising:
a sequence of SEQ ID NO: 2 or a sequence having identity greater than or equal to 90% with SEQ ID NO: 2; and
a transgene placed under the control of said promoter.

6. The expression system according to claim 5, wherein the transgene encodes a protein selected from the group consisting of: dystrophin, HSP-40 homologue B6, calpain 3, dysferlin, sarcoglycan, FKRP (Fukutin-Related Protein) and Anoctamin5.

7. The expression system according to claim 6, wherein the calpain 3 protein has the sequence SEQ ID NO: 8.

8. The expression system according to claim 5, further comprising at least one additional sequence selected from the group consisting of:
a sequence for transcript stabilization;
a polyadenylation signal;
an enhancer sequence; and
a target sequence of a microRNA.

9. The expression system according to claim 8, wherein it further comprises at least one target sequence of miR208a.

10. The expression system according to claim 5, wherein it comprises a viral vector.

11. A pharmaceutical composition comprising an expression system comprising a promoter comprising:
a sequence of SEQ ID NO: 2 or a sequence having identity greater than or equal to 90% with SEQ ID NO: 2; and
a transgene placed under the control of said promoter.

12. (canceled)

13. (canceled)

14. A method of treating a neuromuscular disease, comprising:
administering to a subject in need a pharmaceutical composition comprising an expression system comprising a promoter comprising:
a sequence of SEQ ID NO: 2 or a sequence having identity greater than or equal to 90% with SEQ ID NO: 2; and
a transgene placed under the control of said promoter.

15. The method of claim 14, wherein the composition is administered systemically.

16. The method of claim 14, wherein the disease is selected from the group consisting of muscular dystrophies, congenital muscular dystrophies, congenital myopathies, distal myopathies, myopathies, myotonic syndromes, ion channel muscle diseases, malignant hyperthermia, metabolic myopathies, Duchenne muscular dystrophy (DMD), Becker muscular dystrophy (BMD), limb-girdle muscular dystrophy 2A (LGMD2A), LGMD2B, LGMD2D, LGMD2I, LGMD1D, and LGMD2L.

17. The expression system of claim 6, wherein the dystrophin is microdystrophin, minidystrophin, or quasidystrophin.

18. The expression system of claim 8, wherein the polyadenylation signal comprises a polyA of the transgene, the polyA of SV40, or the polyA of human β globin (HBB2).

19. The expression system of claim 10, wherein the viral vector is an adeno associated viral vector (AAV).

20. The expression system of claim 19, wherein the AAV comprises a capsid selected from the group consisting of AAV8 capsid, AAV9 capsid, AAV9 rh74 capsid, and AAV9 rh74 P1 capsid.


SEQUENCE LISTING (14 sequences)

SEQ ID NO: 1 - DNA, 2239 bp, Artificial Sequence
  Synthetic Polynucleotide
  misc_feature: human ACTA1 promoter -2000 to + 239
tcaaggcccaaatgtaagctagtccccttacgttacatgcagctcatttgctaagtggtttttttctagtatctccactactcgctgacacaggaggacacaggatgttaaaaaggaaatacagttctgtcaattattcacttactctccaaaatacttggaagaactaaatatggaaccataggagactttatcctcaccgcatagtccctatactagtcaaactccttattttttaattgatcatttttaggaaggtagcattttattcactagaacatttttgttaatacttgtttatttttgggatgaactgccatgatgtgggctacagaggagggtcgcatatgcttccatcccccttttagagaatccacacctgtcccagttgctgggttccactaccaaaagtgaattgcaactattttaggagcacttaagcacatccgaaaaatgagtgattctgttctggcccacaccacatcactgatgtacccccttaaagcatgtccctgagttcatcacagaagactgctcctcctgtgccctccacaaggttagaactgtccttgtcttagggaaaaaggagagagagagagagagagagagagagagagagagagagagagagagagagggacaggcaccaactgggtaacctctgctgacccccactctactttaccataagtagctccaaatccttctagaaaatctgaaaggcatagccccatatatcagtgatataaatagaacctgcagcaggctctggtaaatgatgactacaaggtggactgggaggcagcccggccttggcaggcatcatcctctaaatataaagatgagtttgttcagcctttgcagaaggaaaaactgccacccatcctagagtgccgcgtccttgtccccccaccccctccaatttattgggaggaaggaccagctaagcctcatctaggaagagcccctcacccatctccacctccactccaggtctagccagtcctgggttgtgacccttgtctttcagccccaggagagggacacacatagtgccaccaaagaggctgggggagggcctcagcccaccaaaacctggggccagtgcgtcctacaggaggggaaccctcaccccttcaatccctttaggagacccaagggcgctgcgcgtccctgaggcggacagctccgtgtgctcaggctttgcgcctgacaggcctatccccgggagcccccgcgcctcctccccggcgctccgccctcgcctccccccgccagttgtctatcctgcgacagctgcgcgccctccggccgccggtggccctctgtgcggtgggggaaggggtcgacgtggctcagctttttggattcagggagctcgggggtgggaagagagaaatggagttccaggggcgtaaaggagagggagttcgccttccttcccttcctgagactcaggagtgactgcttctccaatcctcccaagcccaccactccacacgactccctcttcccggtagtcgcaagtgggagtttggggatctgagcaaagaacccgaagaggagttgaaatattggaagtcagcagtcaggcaccttcccgagcgcccagggcgctcagagtggacatggttggggaggcctttgggacaggtgcggttcccggagcgcaggcgcacacatgcacccaccggcgaacgcggtgaccctcgccccaccccatcccctccggcgggcaactgggtcgggtcaggaggggcaaacccgctagggagacactccatatacggcccggcccgcgttacctgggaccgggccaacccgctccttctttggtcaacgcaggggacccgggcgggggcccaggccgcgaaccggccgagggagggggctctagtgcccaacacccaaatatggctcgagaagggcagcgacattcctgcggggtggcgcggagggaatgcccgcgggctatataaaacctgagcagagggacaagcggccaccgcagcggacagcgccaagtgaagcctcgcttcccctccgcggcgaccagggcccgagccgagagtagcagttgtagctacccgcccaggtagggcaggagttgggaggggacagggggacagggcactaccgaggggaacctgaaggactccggggcagaacccagtcggttcacctggtcagccccaggcctgcgccctgagcgctgtgcctcgtctccggagccacacgc

SEQ ID NO: 2 - DNA, 324 bp, Artificial Sequence
  Synthetic Polynucleotide
  misc_feature: truncated pACTA1
aaaggcatagccccatatatcagtgatataaatagaacctgcagcaggctctggtaaatgatgactacaaggtggactgggaggcagcccggccttggcaggcatcgaccgggccaacccgctccttctttggtcaacgcaggggacccgggcgggggcccaggccgcgaaccggccgagggagggggctctagtgcccaacacccaaatatggctcgagaagggcagcgacattcctgcggggtggcgcggagggaatgcccgcgggctatataaaacctgagcagagggacaagcggccaccgcagcggacagcgccaagtg

SEQ ID NO: 3 - DNA, 106 bp, Artificial Sequence
  Synthetic Polynucleotide
  misc_feature: part 1 truncated pACTA1 (distal)
aaaggcatagccccatatatcagtgatataaatagaacctgcagcaggctctggtaaatgatgactacaaggtggactgggaggcagcccggccttggcaggcatc

SEQ ID NO: 4 - DNA, 218 bp, Artificial Sequence
  Synthetic Polynucleotide
  misc_feature: part 2 truncated pACTA1 (proximal)
gaccgggccaacccgctccttctttggtcaacgcaggggacccgggcgggggcccaggccgcgaaccggccgagggagggggctctagtgcccaacacccaaatatggctcgagaagggcagcgacattcctgcggggtggcgcggagggaatgcccgcgggctatataaaacctgagcagagggacaagcggccaccgcagcggacagcgccaagtg

SEQ ID NO: 5 - DNA, 1061 bp, Artificial Sequence
  Synthetic Polynucleotide
  misc_feature: human desmin promoter
taccccctgccccccacagctcctctcctgtgccttgtttcccagccatgcgttctcctctataaatacccgctctggtatttggggttggcagctgttgctgccagggagatggttgggttgacatgcggctcctgacaaaacacaaacccctggtgtgtgtgggcgtgggtggtgtgagtagggggatgaatcagggagggggcgggggacccagggggcaggagccacacaaagtctgtgcgggggtgggagcgcacatagcaattggaaactgaaagcttatcagaccctttctggaaatcagcccactgtttataaacttgaggccccaccctcgacagtaccggggaggaagagggcctgcactagtccagagggaaactgaggctcagggctagctcgcccatagacatacatggcaggcaggctttggccaggatccctccgcctgccaggcgtctccctgccctcccttcctgcctagagacccccaccctcaagcctggctggtctttgcctgagacccaaacctcttcgacttcaagagaatatttaggaacaaggtggtttagggcctttcctgggaacaggccttgaccctttaagaaatgacccaaagtctctccttgaccaaaaaggggaccctcaaactaaagggaagcctctcttctgctgtctcccctgaccccactcccccccaccccaggacgaggagataaccagggctgaaagaggcccgcctgggggctgcagacatgcttgctgcctgccctggcgaaggattggcaggcttgcccgtcacaggacccccgctggctgactcaggggcgcaggcctcttgcgggggagctggcctccccgcccccacggccacgggccgccctttcctggcaggacagcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgc

SEQ ID NO: 6 - DNA, 3319 bp, Artificial Sequence
  Synthetic Polynucleotide
  misc_feature: AAV9-truncated pACTA1-GFP-Luc
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacattaattttttgcggccgcaaaggcatagccccatatatcagtgatataaatagaacctgcagcaggctctggtaaatgatgactacaaggtggactgggaggcagcccggccttggcaggcatcgaccgggccaacccgctccttctttggtcaacgcaggggacccgggcgggggcccaggccgcgaaccggccgagggagggggctctagtgcccaacacccaaatatggctcgagaagggcagcgacattcctgcggggtggcgcggagggaatgcccgcgggctatataaaacctgagcagagggacaagcggccaccgcagcggacagcgccaagtggctagcgctaccggtcgtcgcgatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggccggactcagatctcgagctcaagcttcgaattcgaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctcgaagatccaccggatctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgtgtagataagtagcatggcgggttaatcattaactacaaggaacccctagtgatggagttggccactccctctctgcgcgctcgctcgctcactgaggccgggcgaccaaaggtcgcc

SEQ ID NO: 7 - DNA, 4376 bp, Artificial Sequence
  Synthetic Polynucleotide
  misc_feature: AAV9-pACTA1-hCalpain3-2xtarget-miR208a
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctacgcgtatgcataaaggcatagccccatatatcagtgatataaatagaacctgcagcaggctctggtaaatgatgactacaaggtggactgggaggcagcccggccttggcaggcatcgaccgggccaacccgctccttctttggtcaacgcaggggacccgggcgggggcccaggccgcgaaccggccgagggagggggctctagtgcccaacacccaaatatggctcgagaagggcagcgacattcctgcggggtggcgcggagggaatgcccgcgggctatataaaacctgagcagagggacaagcggccaccgcagcggacagcgccaagtggctagcgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttttggttgggataaggctggattattctgagtccaagctaggcccttttgctaatcttgttcatacctcttatcttcctcccacagctcctgggcaacgtgctggtctctgtgctggcccatcactttggcaaagaattcgccaccatgccgaccgtcattagcgcatctgtggctccaaggacagcggctgagccccggtccccagggccagttcctcacccggcccagagcaaggccactgaggctgggggtggaaacccaagtggcatctattcagccatcatcagccgcaattttcctattatcggagtgaaagagaagacattcgagcaacttcacaagaaatgtctagaaaagaaagttctttatgtggaccctgagttcccaccggatgagacctctctcttttatagccagaagttccccatccagttcgtctggaagagacctccggaaatttgcgagaatccccgatttatcattgatggagccaacagaactgacatctgtcaaggagagctaggggactgctggtttctcgcagccattgcctgcctgaccctgaaccagcaccttcttttccgagtcataccccatgatcaaagtttcatcgaaaactacgcagggatcttccacttccagttctggcgctatggagagtgggtggacgtggttatagatgactgcctgccaacgtacaacaatcaactggttttcaccaagtccaaccaccgcaatgagttctggagtgctctgctggagaaggcttatgctaagctccatggttcctacgaagctctgaaaggtgggaacaccacagaggccatggaggacttcacaggaggggtggcagagttttttgagatcagggatgctcctagtgacatgtacaagatcatgaagaaagccatcgagagaggctccctcatgggctgctccattgatgatggcacgaacatgacctatggaacctctccttctggtctgaacatgggggagttgattgcacggatggtaaggaatatggataactcactgctccaggactcagacctcgaccccagaggctcagatgaaagaccgacccggacaatcattccggttcagtatgagacaagaatggcctgcgggctggtcagaggtcacgcctactctgtcacggggctggatgaggtcccgttcaaaggtgagaaagtgaagctggtgcggctgcggaatccgtggggccaggtggagtggaacggttcttggagtgatagatggaaggactggagctttgtggacaaagatgagaaggcccgtctgcagcaccaggtcactgaggatggagagttctggatgtcctatgaggatttcatctaccatttcacaaagttggagatctgcaacctcacggccgatgctctgcagtctgacaagcttcagacctggacagtgtctgtgaacgagggccgctgggtacggggttgctctgccggaggctgccgcaacttcccagatactttctggaccaaccctcagtaccgtctgaagctcctggaggaggacgatgaccctgatgactcggaggtgatttgcagcttcctggtggccctgatgcagaagaaccggcggaaggaccggaagctaggggccagtctcttcaccattggcttcgccatctacgaggttcccaaagagatgcacgggaacaagcagcacctgcagaaggacttcttcctgtacaacgcctccaaggcccgtagcaaaacctacatcaacatgagagaggtgagccagcgcttccgcctgcctcccagcgagtacgtcatcgtgccctccacctacgagccccaccaggagggggaattcatcctccgggtcttctctgaaaagaggaacctctctgaggaagttgaaaataccatctccgtggatcggccagtgaaaaagaaaaaaaccaagcccatcatcttcgtttcggacagagcaaacagcaacaaggagctgggtgtggaccaggagtcagaggagggcaaaggcaaaacaagccctgataagcaaaagcagtccccacagccacagcctggcagctctgatcaggaaagtgaggaacagcaacaattccggaacattttcaagcagatagcaggagatgacatggagatctgtgcagatgagctcaagaaggtccttaacacagtcgtgaacaaacacaaggacctgaagacacacgggttcacactggagtcctgccgtagcatgattgcgctcatggatacagatggctctggaaagctcaacctgcaggagttccaccacctctggaacaagattaaggcctggcagaaaattttcaaacactatgacacagaccagtccggcaccatcaacagctacgagatgcgaaatgcagtcaacgacgcaggattccacctcaacaaccagctctatgacatcattaccatgcggtacgcagacaaacacatgaacatcgactttgacagtttcatctgctgcttcgttaggctggagggcatgttcagagcttttcatgcatttgacaaggatggagatggtatcatcaagctcaacgttctggagtggctgcagctcaccatgtatgcctgacgcgtacaagctttttgctcgtcttatcctaggacaagctttttgctcgtcttatgcggcattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatcaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 8 - protein, 821 aa, Artificial Sequence
  human calpain 3
MPTVISASVAPRTAAEPRSPGPVPHPAQSKATEAGGGNPSGIYSAIISRNFPIIGVKEKTFEQLHKKCLEKKVLYVDPEFPPDETSLFYSQKFPIQFVWKRPPEICENPRFIIDGANRTDICQGELGDCWFLAAIACLTLNQHLLFRVIPHDQSFIENYAGIFHFQFWRYGEWVDVVIDDCLPTYNNQLVFTKSNHRNEFWSALLEKAYAKLHGSYEALKGGNTTEAMEDFTGGVAEFFEIRDAPSDMYKIMKKAIERGSLMGCSIDDGTNMTYGTSPSGLNMGELIARMVRNMDNSLLQDSDLDPRGSDERPTRTIIPVQYETRMACGLVRGHAYSVTGLDEVPFKGEKVKLVRLRNPWGQVEWNGSWSDRWKDWSFVDKDEKARLQHQVTEDGEFWMSYEDFIYHFTKLEICNLTADALQSDKLQTWTVSVNEGRWVRGCSAGGCRNFPDTFWTNPQYRLKLLEEDDDPDDSEVICSFLVALMQKNRRKDRKLGASLFTIGFAIYEVPKEMHGNKQHLQKDFFLYNASKARSKTYINMREVSQRFRLPPSEYVIVPSTYEPHQEGEFILRVFSEKRNLSEEVENTISVDRPVKKKKTKPIIFVSDRANSNKELGVDQESEEGKGKTSPDKQKQSPQPQPGSSDQESEEQQQFRNIFKQIAGDDMEICADELKKVLNTVVNKHKDLKTHGFTLESCRSMIALMDTDGSGKLNLQEFHHLWNKIKAWQKIFKHYDTDQSGTINSYEMRNAVNDAGFHLNNQLYDIITMRYADKHMNIDFDSFICCFVRLEGMFRAFHAFDKDGDGIIKLNVLEWLQLTMYA

SEQ ID NO: 9 - DNA, 16 bp, Artificial Sequence
  Synthetic Polynucleotide
  misc_feature: FWD hcalpain3
cgcctccaaggcccgt

SEQ ID NO: 10 - DNA, 16 bp, Artificial Sequence
  Synthetic Polynucleotide
  misc_feature: REV hcalpain3
ggcggaagcgctggct

SEQ ID NO: 11 - DNA, 21 bp, Artificial Sequence
  Synthetic Polynucleotide
  misc_feature: Probe hcalpain3
ctacatcaacatgagagaggt

SEQ ID NO: 12 - DNA, 20 bp, Artificial Sequence
  Synthetic Polynucleotide
  misc_feature: FWD Rplp0
ctccaagcagatgcagcaga

SEQ ID NO: 13 - DNA, 20 bp, Artificial Sequence
  Synthetic Polynucleotide
  misc_feature: REV Rplp0
atagccttgcgcatcatggt

SEQ ID NO: 14 - DNA, 22 bp, Artificial Sequence
  Synthetic Polynucleotide
  misc_feature: Probe Rplp0
ccgtggtgctgatgggcaagaa